SEQUENCE LISTING (2 sequences)

SEQ ID NO: 1 - protein, 395 aa, Homo sapiens
MSANATLKPLCPILEQMSRLQSHSNTSIRYIDHAAVLLHGLASLLGLVENGVILFVVGCRMRQTVVTTWVLHLALSDLLASASLPFFTYFLAVGHSWELGTTFCKLHSSIFFLNMFASGFLLSAISLDRCLQVVRPVWAQNHRTVAAAHKVCLVLWALAVLNTVPYFVFRDTISRLDGRIMCYYNVLLLNPGPDRDATCNSRQAALAVSKFLLAFLVPLAIIASSHAAVSLRLQHRGRRRPGRFVRLVAAVVAAFALCWGPYHVFSLLEARAHANPGLRPLVWRGLPFVTSLAFFNSVANPVLYVLTCPDMLRKLRRSLRTVLESVLVDDSELGGAGSSRRRRTSSTARSASPLALCSRPEEPRGPARLLGWLLGSCAASPQTGPLNRALSSTSS

SEQ ID NO: 2 - DNA, 1188 bp, Homo sapiens
atgtcggccaacgccacactgaagccactctgccccatcctggagcagatgagccgtctccagagccacagcaacaccagcatccgctacatcgaccacgcggccgtgctgctgcacgggctggcctcgctgctgggcctggtggagaatggagtcatcctcttcgtggtgggctgccgcatgcgccagaccgtggtcaccacctgggtgctgcacctggcgctgtccgacctgttggcctctgcttccctgcccttcttcacctacttcttggccgtgggccactcgtgggagctgggcaccaccttctgcaaactgcactcctccatcttctttctcaacatgttcgccagcggcttcctgctcagcgccatcagcctggaccgctgcctgcaggtggtgcggccggtgtgggcgcagaaccaccgcaccgtggccgcggcgcacaaagtctgcctggtgctttgggcactagcggtgctcaacacggtgccctatttcgtgttccgggacaccatctcgcggctggacgggcgcattatgtgctactacaatgtgctgctcctgaacccggggcctgaccgcgatgccacgtgcaactcgcgccaggcggccctggccgtcagcaagttcctgctggccttcctggtgccgctggcgatcatcgcctcgagccacgcggccgtgagcctgcggttgcagcaccgcggccgccggcggccaggccgcttcgtgcgcctggtggcagccgtcgtggccgccttcgcgctctgctgggggccctaccacgtgttcagcctgctggaggcgcgggcgcacgcaaacccggggctgcggccgctcgtgtggcgcgggctgcccttcgtcaccagcctggccttcttcaacagcgtggccaacccggtgctctacgtgctcacctgccccgacatgctgcgcaagctgcggcgctcgctgcgcacggtgctggagagcgtgctggtggacgacagcgagctgggtggcgcgggaagcagccgccgccgccgcacctcctccaccgcccgctcggcctcccctttagctctctgcagccgcccggaggaaccgcggggccccgcgcgtctcctcggctggctgctgggcagctgcgcagcgtccccgcagacgggccccctgaaccgggcgctgagcagcacctcgagttag

(19) United States
(12) Patent Application Publication
(10) Pub. No.: US 2009/0105218 A1
(43) Pub. Date: Apr. 23, 2009

(54) CRTH2 Receptor Ligands For Therapeutic Use

(75) Inventors: Trond Ulven, Hoersholm (DK); Thomas Frimurer, Hoersholm (DK); Oystein Rist, Hoersholm (DK); Evi Kostenis, Hoersholm (DK); Thomas Hogberg, Hoersholm (DK)

Correspondence Address: BANNER & WITCOFF, LTD., 1100 13th STREET, N.W., SUITE 1200, WASHINGTON, DC, 20005-4051, US

(73) Assignee: 7TM PHARMA A/S, Hoersholm (DK)

(21) Appl. No.: 11/597,938

(22) PCT Filed: May 30, 2005
(86) PCT No.: PCT/EP2005/005883

§ 371 Date: Sep. 16, 2008

(30) Foreign Application Priority Data
May 29, 2004 (GB) 0412198.4
Jun. 24, 2004 (GB) 0414195.8

(51) Int. Cl. A61K 31/196; A61K 31/44; A61K 31/381; A61K 31/341; A61K 31/5375; A61K 31/445; A61K 31/55; A61P 17/00; A61P 25/00; A61P 1/00; A61P 11/00

(52) U.S. Cl. 514/212.01; 514/563; 514/562; 514/352; 514/445; 514/471; 514/238.8; 514/331

(57) ABSTRACT

Compounds of formula (I) are useful in the treatment of disease responsive to modulation of CRTH2 receptor activity, wherein: A represents a carboxyl group —COOH, or a carboxyl bioisostere; L1 is a bond, —CH₂—, —OCH₂—, —CH₂CH₂— or —CH═CH—; L2 is CONH—, —NHCO—, SO₂NR¹—, —NR¹SO₂ wherein R¹ is hydrogen or C₁-C₃ alkyl, or a divalent radical of formula (X) or (Y), wherein ring Q is a non aromatic heterocyclic ring containing 5 to 7 ring atoms, including the nitrogen shown; L3 is a divalent linker radical of formula -(Alk¹)ₘ-(Z)ₙ-(Alk²)ₚ as defined in the description; ring Ar¹ is an optionally substituted divalent phenyl radical or divalent 5- or 6-membered monocyclic heteroaryl radical, in which L1 and the H[B]ₛL3L2Ar²CONH-radical are linked to adjacent ring carbon atoms; ring Ar² is an optionally substituted 1,3-phenylene radical, or an optionally substituted divalent 5- or 6-membered monocyclic heteroaryl radical, in which AL1Ar¹NHCO-radical and the H[B]ₛL3L2-radical are linked to ring carbon atoms which are not in ortho relationship; ring B is as defined for Ar², or an optionally substituted cycloalkyl, N-pyrrolidinyl, N-piperidinyl or N-azepinyl ring; and s is 0 or 1.

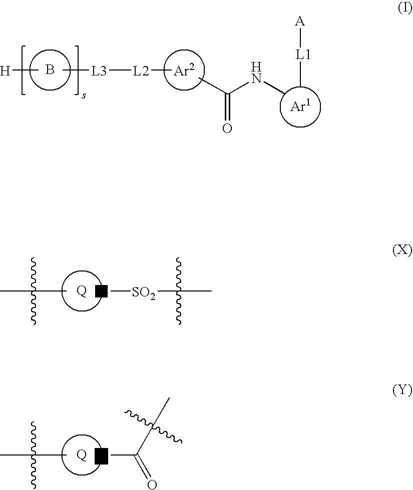

This invention relates to the use of a class of compounds which are ligands of the CRTH2 receptor (Chemoattractant Receptor-homologous molecule expressed on T Helper cells type 2), in the treatment of diseases responsive to modulation of CRTH2 receptor activity, principally diseases having a significant inflammatory component. The invention also relates to novel members of that class of ligands and pharmaceutical compositions containing them.

### BACKGROUND TO THE INVENTION

The natural ligand of the G-protein coupled receptor CRTH2 is prostaglandin D2. As its name implies, CRTH2 is expressed on T helper cells type 2 (TH2 cells) but it is also known to be expressed on eosinophils and basophil cells. Cell activation as a result of binding of PGD2 to the CRTH2 receptor results in a complex biological response, including release of inflammatory mediators. Elevated levels of PGD2 are therefore associated with many diseases which have a strong inflammatory component, such as asthma, rhinitis and allergies. Blocking binding of PGD2 to the CRTH2 receptor is therefore a useful therapeutic strategy for treatment of such diseases.

Some small molecule ligands of CRTH2, apparently acting as antagonists of PGD2, are known, for example as proposed in the following patent publications: WO 03/097042, WO 03/097598, WO 03/066046, WO 03/066047, WO 03/101961, WO 03/101981 GB 2388540, WO 04/089885 and WO 05/018529.

### BRIEF DESCRIPTION OF THE INVENTION

The structures of the PGD2 antagonist compounds referred to in the foregoing publications have a bicyclic or tricyclic core ring system related to the indole core of indomethacin, a known anti-inflammatory agent, now known to bind to CRTH2. The present invention arises from the identification of a class of compounds having a monocyclic core whose substituent moieties are selected and orientated by the monocyclic core to interact with and bind to CRTH2. The class of compounds with which this invention is concerned are thus capable of modulating CRTH2 activity, and are useful in the treatment of diseases which benefit from such modulation, for example asthma, allergy and rhinitis.

### DETAILED DESCRIPTION OF THE INVENTION

According to the present invention, there is provided the use of a compound of formula (I) or a salt, hydrate or solvate thereof in the manufacture of a composition for the treatment of disease responsive to modulation of CRTH2 receptor activity:

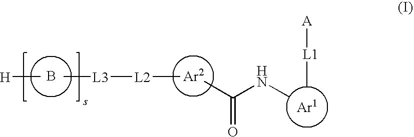

wherein:
A represents a carboxyl group —COOH, or a carboxyl bioisostere,
L1 is a bond, —CH₂—, —OCH₂—, —CH₂CH₂— or —CH═CH—;
L2 is CONH—, —NHCO—, SO₂NR¹—, —NR¹SO₂ wherein R¹ is hydrogen or C₁-C₃ alkyl, or a divalent radical of formula (X) or (Y),

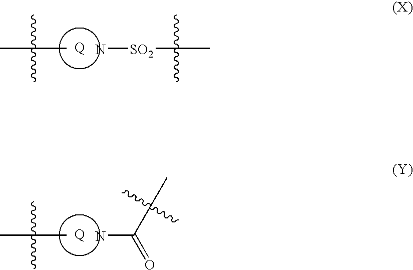

wherein ring Q is a non aromatic heterocyclic ring containing 5 to 7 ring atoms, including the nitrogen shown;
L3 is a divalent radical of formula -(Alk¹)ₘ-(Z)ₙ-(Alk²)ₚ wherein
m, n and p are independently 0 or 1,
Alk¹ and Alk² are independently optionally substituted straight or branched chain C₁-C₃ alkylene or C₂-C₃ alkenylene radicals which may contain a compatible —O—, —S— or —NR-link wherein R is hydrogen or C₁-C₃alkyl, and
Z is —O—; —S—; —C(═O)—; —SO₂—; —SO—; or —NR—, wherein R is hydrogen or C₁-C₃ alkyl; or a divalent 5- or 6-membered monocyclic carbocyclic or heterocyclic radical,
   ring Ar¹ is an optionally substituted divalent phenyl radical or divalent 5- or 6-membered monocyclic heteroaryl radical, in which L1 and the H[B]ₛL3L2ArCONH-radical are linked to adjacent ring carbon atoms;
   ring Ar² is an optionally substituted 1,3-phenylene radical, or an optionally substituted divalent 5- or 6-membered monocyclic heteroaryl radical, in which AL1Ar¹NHCO-radical and the H[B]ₛL3L2-radical are linked to ring carbon atoms which are not in ortho relationship;
   ring B is as defined for Ar², or an optionally substituted cycloalkyl, N-pyrrolidinyl, N-piperidinyl or N-azepinyl ring; and
   s is 0 or 1.

Preferably, in the compounds (I), the total length of L3-L2 and the —CONH— linking Ar¹ and Ar² does not exceed that of an unbranched saturated chain of 10 carbon atoms

In some embodiments, in the compounds (I), (i) the length of each of L3-L2 does not exceed that of an unbranched saturated chain of 5 carbon atoms and (ii) the total length of L3-L2 and the —CONH— linking Ar¹ and Ar² does not exceed that of an unbranched saturated chain of 7 carbon atoms, and (iii) neither of L1, L3-L2 and L4 includes more than two substituents different from hydrogen.

The compounds with which the invention is concerned are defined by reference to formula (I) as a result of studies towards elucidation of the ligand binding site of CRTH2. Such studies led to the overall conclusion that a general pharmacophore comprising one negatively charged moiety, represented by AL1-, and two aromatic and/or hydrophobic moieties, represented by H(B)ₛL3L2Ar²CONH— and Ar¹, oriented in an approximate triangle, would form an arrangement for interaction with the receptor binding site. It was concluded that the substituent groupings AL1-, and H(B)ₛL3L2Ar²CONH— should be on adjacent ring atoms of Ar¹. The linkers L3 L2 and the —CONH— linking Ar¹ and Ar² provide some flexibility to the molecule to facilitate optimum binding. The restrictions on the lengths of, and substitutions in, the linkers L2L4 are in order to restrict the total molecular size and complexity of structures for use in accordance with the invention. For the avoidance of doubt, the length of a radical for the purposes of this description and claims, is the number of connected atoms in the shortest chain of atoms from terminal atom to terminal atom of the radical. Preferably the compounds with which the invention is concerned should have a molecular weight of no more than 600. Optional substituents in any element of the compounds (I) are permitted as in the definition of compounds (I). Such substituents can modulate pharmacokinetic and solubility properties, as well as picking up additional binding interactions with the receptor.

In another aspect, the invention provides a method of treatment of a subject suffering from a disease responsive to modulation of CRTH2 receptor activity, which comprised administering to the subject an amount of a compound (I) as defined and described above effective to ameliorate the disease.

In particular, compounds with which the invention is concerned are useful in the treatment of disease associated with elevated levels of prostaglandin D2 (PGD2) or one or more active metabolites thereof.

Examples of such diseases include asthma, rhinitis, allergic airway syndrome, allergic rhinobronchitis, bronchitis, chronic obstructive pulmonary disease (COPD), nasal polyposis, sarcoidosis, farmer's lung, fibroid lung, cystic fibrosis, chronic cough, conjunctivitis, atopic dermatitis, Alzheimer's disease, amyotrophic lateral sclerosis, AIDS dementia complex, Huntington's disease, frontotemporal dementia, Lewy body dementia, vascular dementia, Guillain-Barre syndrome, chronic demyelinating polyradiculoneurophathy, multifocal motor neuropathy, plexopathy, multiple sclerosis, encephalomyelitis, panencephalitis, cerebellar degeneration and encephalomyelitis, CNS trauma, migraine, stroke, rheumatoid arthritis, ankylosing spondylitis, Behçet's Disease, bursitis, carpal tunnel syndrome, inflammatory bowel disease, Crohn's disease, ulcerative colitis, dermatomyositis, Ehlers-Danlos Syndrome (EDS), fibromyalgia, myofascial pain, osteoarthritis (OA), osteonecrosis, psoriatic arthritis, Reiter's syndrome (reactive arthritis), sarcoidosis, scleroderma, Sjogren's Syndrome, soft tissue disease, Still's Disease, tendinitis, polyarteritis Nodossa, Wegener's Granulomatosis, myositis (polymyositis dermatomyositis), gout, atherosclerosis, lupus erythematosus, systemic lupus erythematosus (SLE), type I diabetes, nephritic syndrome, glomerulonephritis, acute and chronic renal failure, eosinophilia fascitis, hyper IgE syndrome, sepsis, septic shock, ischemic reperfusion injury in the heart, allograft rejection after transplantations, and graft versus host disease.

However, the compounds with which the invention is concerned are primarily of value for the treatment asthma, rhinitis, allergic airway syndrome, and allergic rhinobronchitis.

Many compounds of formula (I) above are novel in their own right, and the invention includes such novel compounds per se.

As used herein, the term “(Cₐ-C_{b})alkyl” wherein a and b are integers refers to a straight or branched chain alkyl radical having from a to b carbon atoms. Thus when a is 1 and b is 6, for example, the term includes methyl, ethyl, n-propyl, isopropyl, n-butyl, isobutyl, sec-butyl, t-butyl, n-pentyl and n-hexyl.

As used herein the term “divalent (Cₐ-C_{b})alkylene radical” wherein a and b are integers refers to a saturated hydrocarbon chain having from a to b carbon atoms and two unsatisfied valences.

As used herein the term “(Cₐ-C_{b})alkenyl” wherein a and b are integers refers to a straight or branched chain alkenyl moiety having from a to b carbon atoms having at least one double bond of either E or Z stereochemistry where applicable. The term includes, for example, vinyl, allyl, 1- and 2-butenyl and 2-methyl-2-propenyl.

As used herein the term “divalent (Cₐ-C_{b})alkenylene radical” means a hydrocarbon chain having from a to a carbon atoms, at least one double bond, and two unsatisfied valences.

As used herein the term “Cₐ-C_{b} alkynyl” wherein a and b are integers refers to straight chain or branched chain hydrocarbon groups having from two to six carbon atoms and having in addition one triple bond. This term would include for example, ethynyl, 1- and 2-propynyl, 1-, 2- and 3-butynyl, 1,2-, 3- and 4-pentynyl, 1-, 2-, 3-, 4- and 5-hexynyl, 3-methyl-1-butynyl, 1-methyl-2-pentynyl.

As used herein the term “divalent (Cₐ-C_{b})alkynylene radical” wherein a and b are integers refers to a divalent hydrocarbon chain having from 2 to 6 carbon atoms, at least one triple bond, and two unsatisfied valences.

As used herein the term “carbocyclic” refers to a mono-, bi- or tricyclic radical having up to 16 ring atoms, all of which are carbon, and includes aryl and cycloalkyl.

As used herein the term “cycloalkyl” refers to a monocyclic saturated carbocyclic radical having from 3-8 carbon atoms and includes, for example, cyclopropyl, cyclobutyl, cyclopentyl, cyclohexyl, cycloheptyl and cyclooctyl.

As used herein the unqualified term “aryl” refers to a mono-, bi- or tri-cyclic carbocyclic aromatic radical, and includes radicals having two monocyclic carbocyclic aromatic rings which are directly linked by a covalent bond. Illustrative of such radicals are phenyl, biphenyl and napthyl.

As used herein the unqualified term “heteroaryl” refers to a mono-, bi- or tri-cyclic aromatic radical containing one or more heteroatoms selected from S, N and O, and includes radicals having two such monocyclic rings, or one such monocyclic ring and one monocyclic aryl ring, which are directly linked by a covalent bond. Illustrative of such radicals are thienyl, benzthienyl, furyl, benzfuryl, pyrrolyl, imidazolyl, benzimidazolyl, thiazolyl, benzthiazolyl, isothiazolyl, benzisothiazolyl, pyrazolyl, oxazolyl, benzoxazolyl, isoxazolyl, benzisoxazolyl, isothiazolyl, triazolyl, benztriazolyl, thiadiazolyl, oxadiazolyl, pyridinyl, pyridazinyl, pyrimidinyl, pyridazinyl, triazinyl, indolyl and indazolyl.

As used herein the unqualified term “heterocyclyl” or “heterocyclic” includes “heteroaryl” as defined above, and in addition means a mono-, bi- or tri-cyclic non-aromatic radical containing one or more heteroatoms selected from S, N and O, and to groups consisting of a monocyclic non-aromatic radical containing one or more such heteroatoms which is covalently linked to another such radical or to a monocyclic carbocyclic radical. Illustrative of such radicals are pyrrolyl, furanyl, thienyl, piperidinyl, imidazolyl, oxazolyl, isoxazolyl, thiazolyl, thiadiazolyl, pyrazolyl, pyridinyl, pyrrolidinyl, pyrimidinyl, morpholinyl, piperazinyl, indolyl, morpholinyl, benzfuranyl, pyranyl, isoxazolyl, benzimidazolyl, methylenedioxyphenyl, ethylenedioxyphenyl, maleimido and succinimido groups.

The term “carboxyl bioisostere” is a term familiar to medicinal chemists (see for example “The Organic Chemistry of Drug Design and Drug Action”, by Richard B. Silverman, pub. Academic Press, 1992), and refers to a group which has similar acid-base characteristics to those of a carboxyl group. Well known carboxyl bioisosteres include —SO₂NHR or —P(═O)(OH)(OR) wherein R is, for example, hydrogen methyl or ethyl, —SO₂OH, —P(═O)(OH)(NH₂), —C(═O)NHCN and groups of formulae:

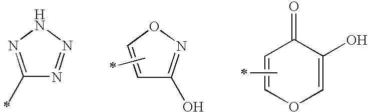

Unless otherwise specified in the context in which it occurs, the term “substituted” as applied to any moiety herein means substituted with up to four compatible substituents, each of which independently may be, for example, (C₁-C₆)alkyl, (C₁-C₆)alkoxy, hydroxy, hydroxy(C₁-C₆)alkyl, mercapto, mercapto(C₁-C₆)alkyl, (C₁-C₆)alkylthio, halo (including fluoro, bromo and chloro), fully or partially fluorinated (C₁-C₃)alkyl, (C₁-C₃)alkoxy or (C₁-C₃)alkylthio such as trifluoromethyl, trifluoromethoxy, and trifluoromethylthio, nitro, nitrile (—CN), oxo, phenyl, phenoxy, monocyclic heteroaryl or heteroaryloxy with 5 or 6 ring atoms, —COOR^{A}, —COR^{A}, —OCOR^{A}, —SO₂R^{A}, —CONR^{A}R^{B}, —SO₂NR^{A}R^{B}, —NR^{A}R^{B}, OCONR^{A}R^{B}, —NR^{B}COR^{A}, NR^{B}COOR^{A}, —NR^{B}SO₂OR^{A} or —NR^{A}CONR^{A}R^{B} wherein R^{A} and R^{B} are independently hydrogen or a (C₁-C₆)alkyl group or, in the case where R^{A} and R^{B} are linked to the same N atom, R^{A} and R^{B} taken together with that nitrogen may form a cyclic amino ring. Where the substituent is phenyl, phenoxy or monocyclic heteroaryl or heteroaryloxy with 5 or 6 ring atoms, the phenyl or heteroaryl ring thereof may itself be substituted by any of the above substituents except phenyl phenoxy, heteroaryl or heteroaryloxy. An “optional substituent” may be one of the foregoing substituent groups.

As used herein the term “salt” includes base addition, acid addition and quaternary salts. Compounds of the invention which are acidic can form salts, including pharmaceutically acceptable salts, with bases such as alkali metal hydroxides, e.g. sodium and potassium hydroxides; alkaline earth metal hydroxides e.g. calcium, barium and magnesium hydroxides; with organic bases e.g. N-methyl-D-glucamine, choline tris(hydroxymethyl)amino-methane, L-arginine, L-lysine, N-ethyl piperidine, dibenzylamine and the like. Those compounds (I) which are basic can form salts, including pharmaceutically acceptable salts with inorganic acids, e.g. with hydrohalic acids such as hydrochloric or hydrobromic acids, sulphuric acid, nitric acid or phosphoric acid and the like, and with organic acids e.g. with acetic, tartaric, succinic, fumaric, maleic, malic, salicylic, citric, methanesulphonic, p-toluenesulphonic, benzoic, benzenesunfonic, glutamic, lactic, and mandelic acids and the like.

Compounds with which the invention is concerned which may exist in one or more stereoisomeric form, because of the presence of asymmetric atoms or rotational restrictions, can exist as a number of stereoisomers with R or S stereochemistry at each chiral centre or as atropisomeres with R or S stereochemistry at each chiral axis. The invention includes all such enantiomers and diastereoisomers and mixtures thereof.

Use of prodrugs, such as esters, of compounds (I) with which the invention is concerned is also part of the invention.

For use in accordance with the above broad aspect of the invention the following structural characteristics are may be present, in any compatible combination, in the compounds (I):
Ar¹ may be an optionally substituted phenyl ring or a ring selected from the group consisting of:

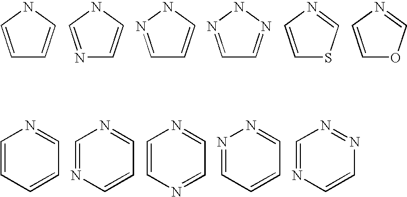

any of which being optionally substituted, for example by one or more selected from fluoro, chloro, bromo, iodo, cyano, nitro, trifluoromethyl, trifluoromethoxy, trifluoromethylthio, (C₁-C₃alkyl)SO₂—, NH₂SO₂—, (C₁-C₃alkyl)NHSO₂—, (C₁-C₃alkyl)₂NSO₂—, C₁-C₆alkyl, C₁-C₆alkoxy, cycloalkyl, aryl, aryloxy, aryl(C₁-C₆) and aryl(C₁-C₆ alkoxy)-;
A may be —COOH or a carboxyl bioisostere selected from —SO₂NHR and —P(═O)(OH)(OR) wherein R is hydrogen methyl or ethyl, —SO₂OH, —P(═O)(OH)(NH₂), —C(═O)NHCN and groups of formulae:

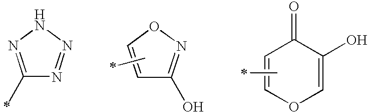

Currently it is preferred that A be —COOH, ie L1 represents a bond.
Ar² may be an optionally substituted 1,3-phenylene radical or may be selected from the following radicals, in either orientation in the case of non-symmetrical radicals:

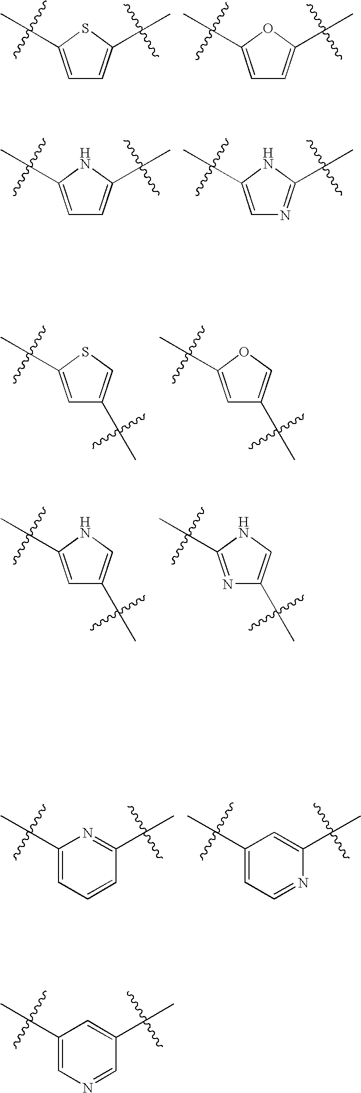

Any optional substituents in ring Ar² may be selected from fluoro, chloro, bromo, iodo, cyano, nitro, trifluoromethyl, trifluoromethoxy, trifluoromethylthio, (C₁-C₃alkyl)SO₂—, NH₂SO₂—, (C₁-C₃alkyl)NHSO₂—, (C₁-C₃alkyl)₂NSO₂—, C₁-C₆ alkyl, C₁-C₆ alkoxy, cycloalkyl, aryl, aryloxy, aryl(C₁-C₆) and aryl(C₁-C₆ alkoxy)-.
s may be 1 and ring B may be an optionally substituted phenyl, thienyl, furanyl, pyridyl, or N-pyrrolidinyl, N-piperidinyl, N-piperazinyl, N-morpholinyl or N-azepinyl ring, and any optional substituents in ring B may be selected from fluoro, chloro, bromo, iodo, cyano, nitro, trifluoromethyl, trifluoromethoxy, trifluoromethylthio, (C₁-C₃alkyl)SO₂—, NH₂SO₂—, (C₁-C₃alkyl)NHSO₂—, (C₁-C₃alkyl)₂NSO₂—, C₁-C₆alkyl, C₁-C₆alkoxy, cycloalkyl, aryl, aryloxy, aryl(C₁-C₆)— or aryl(C₁-C₆ alkoxy)-.

When L2 is a divalent radical of formula (X) or (Y) wherein the divalent radical -Q- may be selected from the following

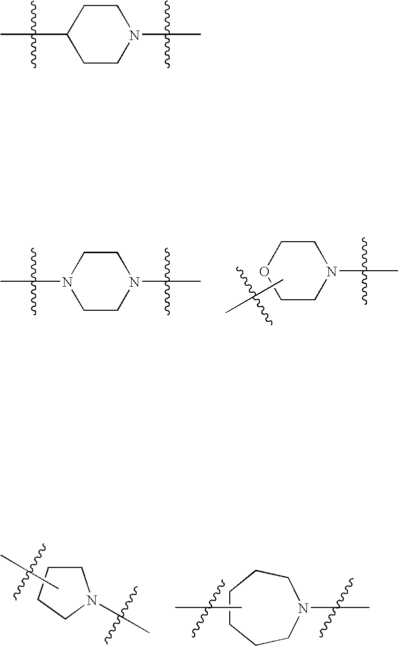

L3 may be a bond or a linker radical selected from —CH₂—, —CH(Ph)- wherein Ph is phenyl, —NR—, —CH₂CH₂—, —CH₂CH₂CH₂—, —CH₂Z-, -ZCH₂—, —CH₂CH₂Z-, —CH₂ZCH₂—, -ZCH₂CH₂—, —CH═CH—, —CH═C(CH₃)—, —CH═N—, —N═CH—, —CH═CHCH₂—, —N═CHCH₂—, —CH═NCH₂—, —CH₂CH═CH—, —CH₂Z-, -ZCH₂—, —CH₂CH₂Z-, —CH₂ZCH₂—, -ZCH₂CH₂—, —CH═CHZ-, and -ZCH═CH— wherein Z is —O—, —S— or —NR— wherein R is hydrogen or C₁-C₃alkyl, any of which radicals being optionally substituted on one of the carbon atoms shown. In many embodiments L3 may be a bond or a linker radical selected from —OCH₂—, —CH₂CH₂—, —CH═CH—, —CH═C(CH₃)—, —NH—, —CH₂OCH₂CH₂—, —CH(Ph) wherein Ph is phenyl, or —CH₂SCH₂—.

One class of compounds for use in accordance with the broad aspect of the invention compound (I) has formula (II):

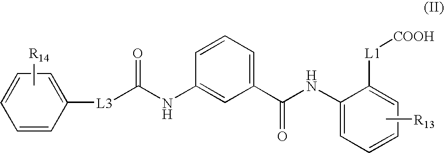

wherein L1 and L3 are as defined in claim 1, and R₁₃ and R₁₄ represent one or more optional substituents in their respective phenyl rings. In such compounds (II), L3 may be a bond or a linker radical selected from —CH₂—, —CH(Ph)- wherein Ph is phenyl, —NR—, —CH₂CH₂—, —CH₂CH₂CH₂—, —CH₂Z-, -ZCH₂—, —CH₂CH₂Z-, —CH₂ZCH₂—, -ZCH₂CH₂—, —CH═CH—, —CH═C(CH₃)—, —CH═N—, —N═CH—, —CH═CHCH₂—, —N═CHCH₂—, —CH═NCH₂—, —CH₂CH═CH—, —CH₂Z-, -ZCH₂—, —CH₂CH₂Z-, —CH₂ZCH₂—, -ZCH₂CH₂—, —CH═CHZ-, and -ZCH═CH— wherein Z is —O—, —S— or —NR— wherein R is hydrogen or C₁-C₃alkyl, any of which radicals being optionally substituted on one of the carbon atoms shown. In may such cases, L3 may be a bond or a linker radical selected from —OCH₂—, —CH₂CH₂—, —CH═CH—, —CH═C(CH₃)—, —NH—, —CH₂OCH₂CH₂—, —CH(Ph) wherein Ph is phenyl, or —CH₂SCH₂—.

Also in the compounds (II), optional substituents R₁₃ and R₁₄ may be selected from fluoro, chloro, bromo, iodo, cyano, nitro, trifluoromethyl, trifluoromethoxy, trifluoromethylthio, (C₁-C₃alkyl)SO₂—, NH₂SO₂—, (C₁-C₃alkyl)NHSO₂—, (C₁-C₃alkyl)₂NSO₂—, C₁-C₆ alkyl, C₁-C₆ alkoxy, cycloalkyl, aryl, aryloxy, aryl(C₁-C₆) and aryl(C₁-C₆ alkoxy)-.

In a narrower aspect, the invention provides compounds, believed to be novel per se, of formula (III), or a salt, hydrate or solvate thereof

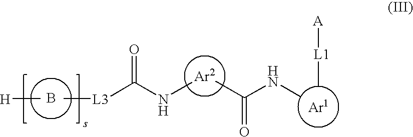

wherein: A, L1 Ar1, Ar2, s and B independently are as defined and discussed above, and L3 is a linker radical selected from —CH₂CH₂—, —CH₂CH₂CH₂—, —CH₂Z-, -ZCH₂—, —CH₂CH₂Z-, —CH₂ZCH₂—, -ZCH₂CH₂—, —CH═CH—, —CH═C(CH₃)—, —CH═N—N═CH—, —CH═CHCH₂—, —N═CHCH₂—, —CH═NCH₂—, —CH₂CH═CH—, —CH₂Z-, -ZCH₂—, —CH₂CH₂Z-, —CH₂ZCH₂—, -ZCH₂CH₂—, —CH═CHZ-, and -ZCH═CH— wherein Z is —O—, —S— or —NR— wherein R is hydrogen or C₁-C₃ alkyl, any of which radicals being optionally substituted on one of the carbon atoms shown,

### Provided That

when A is —COOH, L1 is a bond, and s is 1 then (a) when Ar² is a 1,3-phenylene radical H—B-L3- is not a radical of formula (C), (D), (E) or (F):

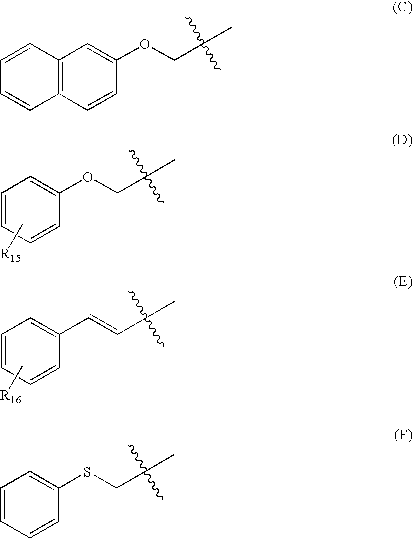

wherein R₁₅ represents hydrogen or 2- or 4-nitro, 2-, 3- or 4-methyl, 2,3-, 2,6-, or 3,4-dimethyl, 2- or 3-methoxy, 2-chloro, 4-bromo, 4-isopropyl, or 4-(1-methylpropyl), R₁₆ represents 4-nitro or 2-methoxy-5-bromo; and (b) when Ar² is a 4-methyl-1,3-phenylene radical H—B-Alk¹- is not a radical of formula (J) or (K)

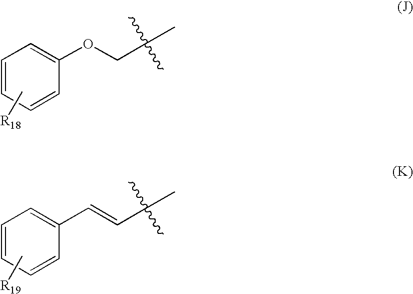

wherein R₁₈ is 2-methoxy and R₁₉ is 2-methoxy-5-bromo; and (c) when Ar² is a 4-methyl-1,3-phenylene radical H—B-Alk¹- is not a radical of formula (L)

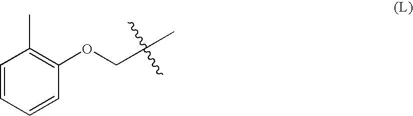

A particular subgroup of compounds (III) consists of those having formula (II), the said formula (II) being subject to the Provisos in the definition of compounds (III),

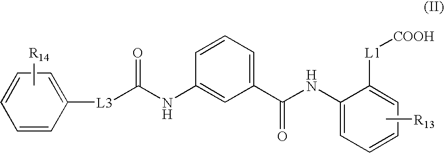

wherein L1 and L3 are as defined in relation to formula (III), and R₁₃ and R₁₄ represent one or more optional substituents in their respective phenyl rings.

In compounds (II), optional substituents R₁₃ and R₁₄ may be selected from fluoro, chloro, bromo, iodo, cyano, nitro, trifluoromethyl, trifluoromethoxy, trifluoromethylthio, (C₁-C₃alkyl)SO₂—, NH₂SO₂—, (C₁-C₃alkyl)NHSO₂—, (C₁-C₃alkyl)₂NSO₂—, C₁-C₆alkyl, C₁-C₆ alkoxy, cycloalkyl, aryl, aryloxy, aryl(C₁-C₆) and aryl(C₁-C₆ alkoxy)-.

The invention also includes a pharmaceutical composition comprising a novel compound of formula (III) or (II) as defined above, together with a pharmaceutically acceptable carrier.

### Compositions

As mentioned above, the compounds with which the invention is concerned are capable of modulating CRTH2 activity, and are useful in the treatment of diseases which benefit from such modulation. Examples of such diseases are referred to above, and include asthma, allergy and rhinitis.

It will be understood that the specific dose level for any particular patient will depend upon a variety of factors including the activity of the specific compound employed, the age, body weight, general health, sex, diet, time of administration, route of administration, rate of excretion, drug combination and the severity of the particular disease undergoing treatment. Optimum dose levels and frequency of dosing will be determined by clinical trial, as is required in the pharmaceutical art.

The compounds with which the invention is concerned may be prepared for administration by any route consistent with their pharmacokinetic properties. The orally administrable compositions may be in the form of tablets, capsules, powders, granules, lozenges, liquid or gel preparations, such as oral, topical, or sterile parenteral solutions or suspensions. Tablets and capsules for oral administration may be in unit dose presentation form, and may contain conventional excipients such as binding agents, for example syrup, acacia, gelatin, sorbitol, tragacanth, or polyvinyl-pyrrolidone; fillers for example lactose, sugar, maize-starch, calcium phosphate, sorbitol or glycine; tabletting lubricant, for example magnesium stearate, talc, polyethylene glycol or silica; disintegrants for example potato starch, or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives such as suspending agents, for example sorbitol, syrup, methyl cellulose, glucose syrup, gelatin hydrogenated edible fats; emulsifying agents, for example lecithin, sorbitan monooleate, or acacia; non-aqueous vehicles (which may include edible oils), for example almond oil, fractionated coconut oil, oily esters such as glycerine propylene glycol, or ethyl alcohol; preservatives, for example methyl or propyl p-hydroxybenzoate or sorbic acid, and if desired conventional flavouring or colouring agents.

For topical application to the skin, the drug may be made up into a cream, lotion or ointment. Cream or ointment formulations which may be used for the drug are conventional formulations well known in the art, for example as described in standard textbooks of pharmaceutics such as the British Pharmacopoeia.

For topical application to the eye, the drug may be made up into a solution or suspension in a suitable sterile aqueous or non aqueous vehicle. Additives, for instance buffers such as sodium metabisulphite or disodium edeate; preservatives including bactericidal and fungicidal agents such as phenyl mercuric acetate or nitrate, benzalkonium chloride or chlorhexidine, and thickening agents such as hypromellose may also be included.

The drug may also be formulated for inhalation, for example as a nasal spray, or dry powder or aerosol inhalers.

The active ingredient may also be administered parenterally in a sterile medium. Depending on the vehicle and concentration used, the drug can either be suspended or dissolved in the vehicle. Advantageously, adjuvants such as a local anaesthetic, preservative and buffering agents can be dissolved in the vehicle.

The compounds with which the invention is concerned may be administered alone, or as part of a combination therapy with other drugs used for treatment of diseases with a major inflammatory component. In the case of asthma, rhinitis, and allergic airway syndrome such drugs include corticosteroids, long-acting inhaled beta-agonists, beta agonists, cromolyn, nedocromil, theophylline, leukotriene receptor antagonists, antihistamines, and anticholinergics (e.g. ipratropium), and are often administered as nasal sprays, dry powder or aerosol inhalers.

In the case of arthritis and related inflammatory diseases other known drugs include glucocorticoids, NSAIDs (Non Steroidal Anti-Inflammatory Drugs—conventional prostaglandin synthesis inhibitors, COX-2 inhibitors, salicylates), and DMARDs (disease-modifying anti-rheumatic drugs such as methotrexate, sulfasalazine, gold, cyclosporine).

### Synthetic Routes

There are multiple synthetic strategies for the synthesis of the compounds (I) with which the present invention is concerned, but all rely on known chemistry, known to the synthetic organic chemist. Thus, compounds according to formula (I) can be synthesised according to procedures described in the standard literature and are well-known to the one skilled in the art. Typical literature sources are “Advanced organic chemistry”, 4^{th} Edition (Wiley), J March, “Comprehensive Organic Transformation”, 2^{nd} Edition (Wiley), R. C. Larock, “Handbook of Heterocyclic Chemistry”, 2^{nd} Edition (Pergamon), A. R. Katritzky), review articles such as found in “Synthesis”, “Acc. Chem. Res.”, “Chem. Rev”, or primary literature sources identified by standard literature searches online or from secondary sources such as “Chemical Abstracts” or “Beilstein”.

Compounds (I) may be formed by amide formation coupling reaction between a carboxylic acid H[B]ₛL3L4Ar²COOH and an amine H₂NAr¹L1A

In an analogous manner the compounds of formula (I) can be made by forming the linker amide, reverse amide, sulfonamide or reverse sulfonamide bond in L2, by typical coupling reactions.

Furthermore, the Ar¹ moiety can also be assembled via ring cyclisation reactions with reactants containing the L1 and L2 units either containing the full appendices as outlined below

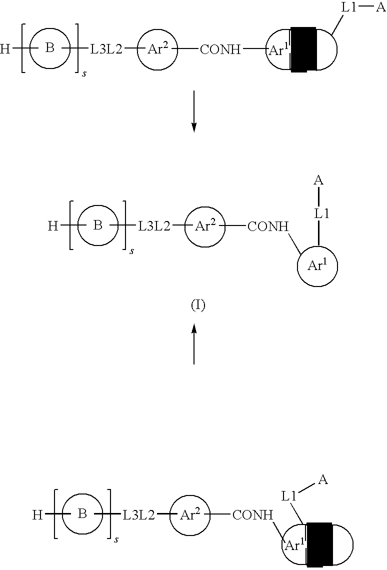

or in forms that can be further functionalised into the final formula (I) structures. For example, 1,2,4-triazoles can be made from acylhydrazides and amides or thioamides; 1,2,4-oxadiazoles from amidoximes and carboxylic esters; 1,3,4-oxadiazoles from acylhydrazides and carboxylic esters; thiazoles from thioamides and □-haloketones; pyridines via various cycloaddition reactions.

The building blocks used in the reactions are either commercially available or made according to standard procedures well-know to one skilled in the art as described in “Advanced organic chemistry”, 4^{th} Edition (Wiley), J March, “Comprehensive Organic Transformation”, 2^{nd} Edition (Wiley), R. C. Larock, “Handbook of Heterocyclic Chemistry”, 2^{nd} Edition (Pergamon), A. R. Katritzky or other suitable literature sources. The Examples herein describe specific strategies for the synthesis of the compounds of the first, second and third preferred sub-classes described above. Analogous compounds are accessible by variation of the intermediates used in the Examples.

The following Examples illustrate the preparation of compounds with which this invention is concerned. Some compounds were synthesised, and some were acquired from commercial sources. In the Examples:

### General Comments:

NMR spectra were obtained on a Bruker Avance AMX 300 MHz instrument. LC/MS was performed on an Agilent 1100-series instrument. LC/MS methods are as follows: An10p8: Column: XTerra MS C18; Flow: 1.0 mL/min; Gradient: 0-5 min: 15-100% MeCN in water, 5-72 min: 100% MeCN; Modifier: 5 mM ammonium formate; MS-ionisation mode: API-ES (pos.). An10n8: Column: XTerra MS C18; Flow: 1.0 mL/min; Gradient: 0-5 min: 15-100% MeCN in water, 5-7½ min: 100% MeCN; Modifier: 5 mM ammonium formate; MS-ionisation mode: API-ES (neg.).

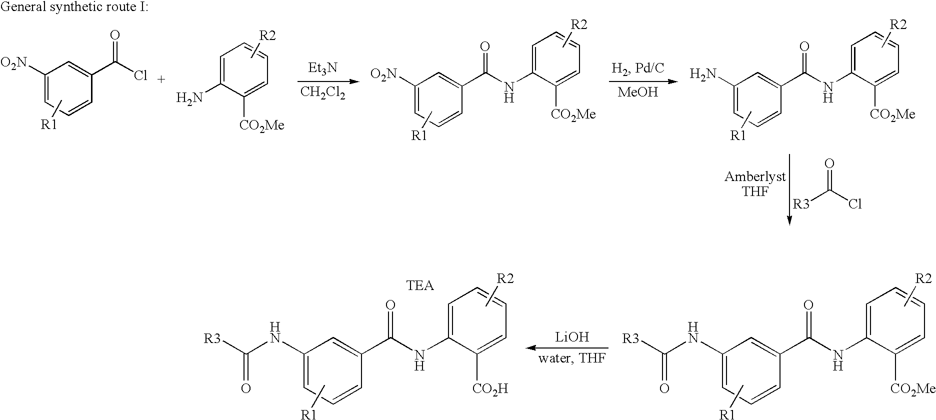

### General Procedure 1 (GP1):

A vial with methyl 2-(3-aminobenzoylamino)benzoate or a substituted derivatives (0.12 mmol) and dry amberlite IRA-68 (220 mg) was added dry THF (1.5 ml), and the mixture was stirred for 10 min. The acylating agent (acyl chloride, sulfonyl chloride or isocyanate) (0.12 mmol) was added, and the reaction mixture was stirred under argon. After 12 h the reaction mixture was added water (0.5 mL) and stirred for 1 h. To the reaction was added LiOH (18 mg) in water (0.2 mL) and stirred at room temperature. After 12 h 1 M HCl (1 mL) was added, and the reaction mixture was extracted with DCM. The organic phase was dried (MgSO₄) and concentrated to give the product, which was purified, if necessary.

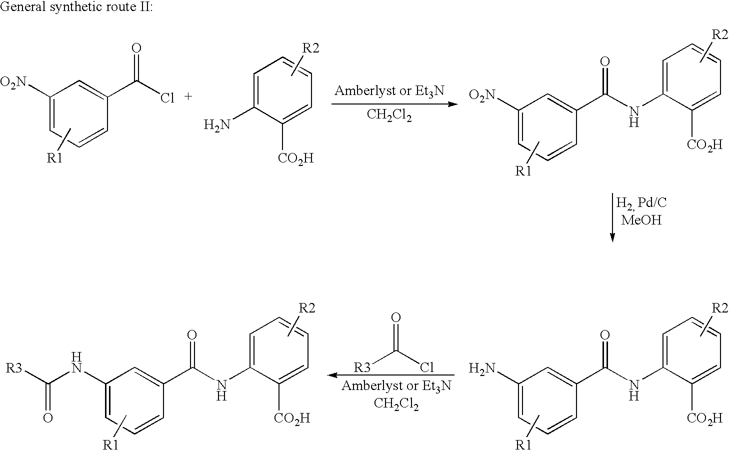

### General Procedure 2 (GP2):

A vial with 2-(3-aminobenzoylamino)benzoaic acid or a substituted derivatives (0.12 mmol) and dry amberlite IRA-68 (220 mg) was added dry CH₂Cl₂ (1.5 mL), and the mixture was stirred for 10 min. The acylating agent (acyl chloride, sulfonyl chloride or isocyanate) (0.12 mmol) was added, and the reaction mixture was stirred under argon. After 12 h the reaction mixture was added PS-trisamine (500 mg) and stirred at room temperature. After another 12 h the resin was filtered off, washed with CH₂Cl₂ and extracted with TFA/CH₂Cl₂ (1:1) (2 mL). The extract was concentrated, and the residue was purified if necessary.

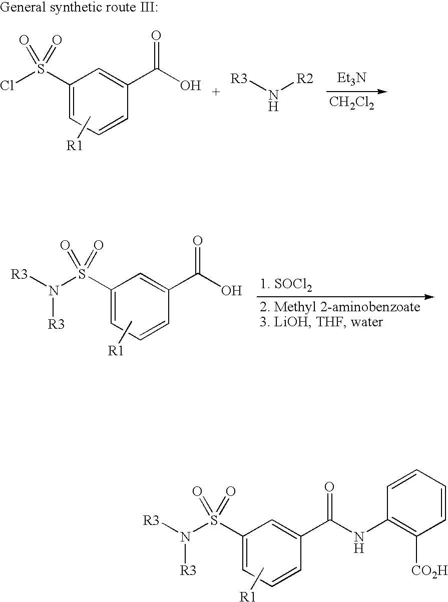

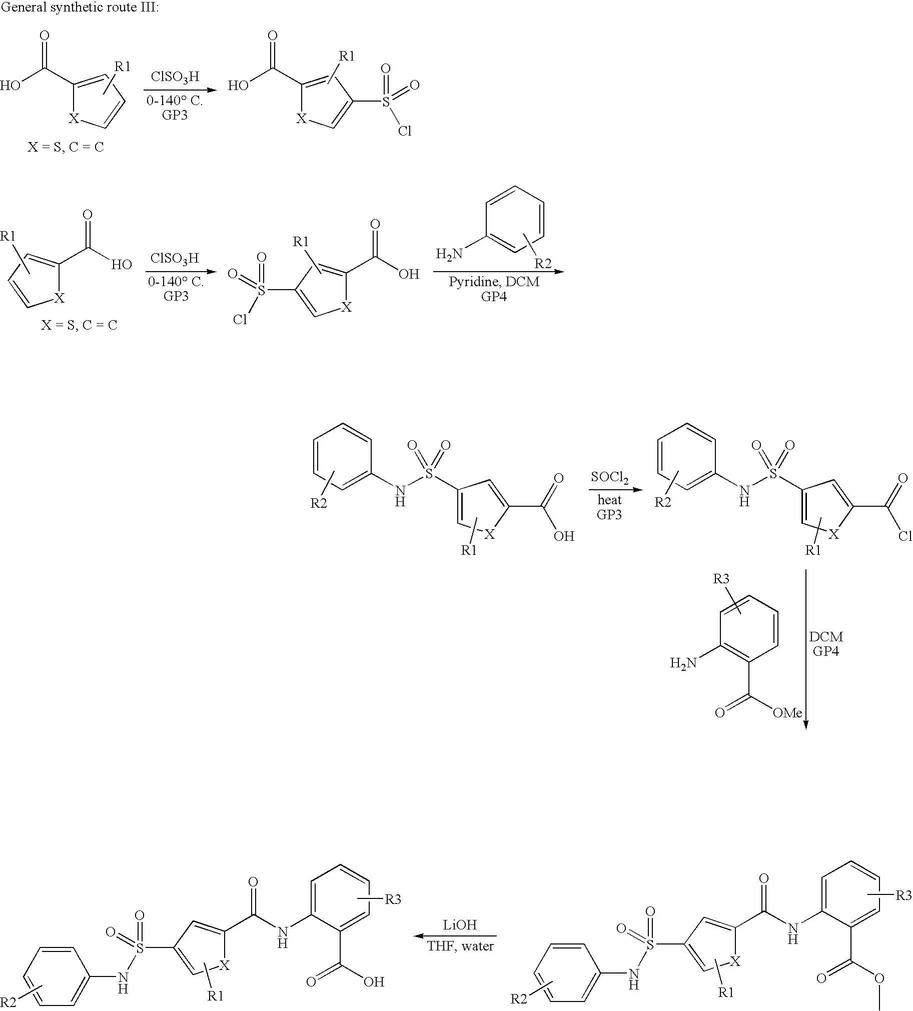

### General Procedure 3 (GP3):

### Chlorosulfonation

On cooling with an ice bath, the aromatic carboxylic acid (100 mmol) was added portionwise to chlorosulfonic acid (54 mL, 800 mmol) at such a rate that the temperature was kept below 10° C. The resulting mixture was allowed to reach room temperature and then heated at an oil bath to 140° C. for 5 h. After cooling to room temperature the mixture was added dropwise to stirred ice-water (250 mL), and stirring was continued for 30 min. The precipitate was collected by filtration and washed with ice water to give the product, which was used directly in the next step.

### General Procedure 4 (GP4):

### Sulfonamide Formation

The aniline (0.42 mmol) was dissolved in dry dichloromethane (3 mL) and pyridine (70 μL) was added. The reaction mixture was stirred for 10 min at room temperature. The sulfonyl chloride (0.42 mmol) was added at 0° C. After stirring at room temperature for 2 days, 1 N HCl was added until pH<1 and the mixture was extracted with dichloromethane. The organic phase was dried (MgSO₄) and concentrated and the product was used directly in the next step.

### General Procedure 5 (GP5):

### Acid Chloride Formation

The carboxylic acid was suspended in thionyl chloride (5 mL). After stirring at 80° C. for 2 h, excess of thionyl chloride was evaporated at 50° C. The residue was stripped with dichloromethane. The product was used directly in the next step.

### General Procedure 6 (GP6):

### Amide Formation

The acid chloride was dissolved in dichloromethane (2 mL) and the aniline (1 equivalent ˜0.3-0.4 mmol) was added slowly. The mixture was stirred at room temperature for 1-2 days. In some cases a precipitate was formed and this was collected and washed with dichloromethane. In cases where the product did not precipitate the mixture was acidified with 1 N HCl until pH<1 and extracted with dichloromethane. The organic phase was dried (MgSO₄) and concentrated and the product was used directly in the next step.

### General Procedure 7 (GP7):

### Ester Hydrolysis

The ester was dissolved in THF/H₂O (3 mL/0.5 mL). Lithium hydroxide monohydrate (0.4 mmol) was added. After stirring at room temperature over night 1 N HCl was added until pH<1. In some cases a precipitate was formed and this was collected and washed with diethyl ether and recrystallized from heptane/ethyl acetate. In cases where the product did not precipitate the mixture was acidified with 1 N HCl until pH<1 and extracted with dichloromethane. The organic phase was dried (MgSO₄) and concentrated. Finally the product was purified on a 1 g SAX Acetate SPE column (equilibrated with MeOH and then eluted with 10% AcOH in MeOH). Overall yield (GP4, GP5, GP6, and GP7) of the final products were 11-39%.

### Intermediate

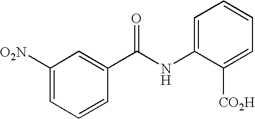

2-(3-Nitrobenzoylamino)benzoic acid. A suspension of 2-aminobenzoic acid (10.1 g, 72 mmol) and Et₃N (31 mL, 228 mmol) in CH₂Cl₂ (500 mL) was added 3-nitrobenzoyl chloride (14.4 g, 76 mmol), and the reaction mixture was stirred under argon. After 12 h the mixture was added 1 M HCl, and the resulting precipitate was filtered off, washed with CH₂Cl₂ and water, and purified by recrystalisation from EtOAc to give 12.13 g (59%) white solid: ¹H NMR (DMSO-d₆): δ 7.2 (m, 1H), 7.6-8.0 (m, 3H), 8.4 (dd, 2H), 8.6 (dd, 2H), 12.31 (s, 1H).

### Intermediate

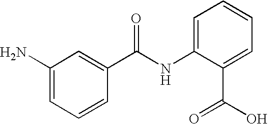

2-(3-Aminobenzoylamino)benzoic acid. A suspension of 2-(3-nitrobenzoylamino)benzoic acid (5.0 g, 18 mmol) and Pd/C (1.6 g) in methanol (200 mL) was stirred under hydrogen (1 atm) for 24 h, then filtered though a pad of celite and concentrated to give 3.23 g (72%) pale yellow solid: LC/MS (an10n8): Rt 2.04 min, m/z 255.0 [M−H]⁻.

### Intermediate

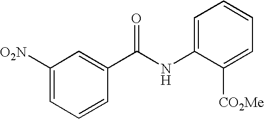

Methyl 2-(3-nitrobenzoylamino)benzoate. A suspension of methyl 2-aminobenzoic acid (11.0 g, 72 mmol) and Et₃N (31 mL, 228 mmol) in CH₂Cl₂ (500 mL) was added 3-nitrobenzoyl chloride (14.4 g, 76 mmol), and the reaction mixture was stirred under argon. After 12 h the mixture was washed with 1 M HCl, the organic layer was dried (MgSO₄) and concentrated, and the precipitate was recrystallized from EtOAc:heptan (1:5) to give 15.74 g (73%) white solid.

LC-MS (an10n8.m): Rt 4.81 min, m/z 299.0 [M−H]⁻.

### Intermediate

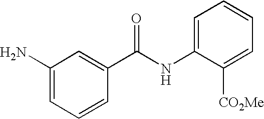

Methyl 2-(3-aminobenzoylamino)benzoate. A suspension of methyl 2-(3-nitrobenzoylamino)-benzoate (5.0 g, 17 mmol) and Pd/C (1.6 g) in methanol (200 mL) was stirred under hydrogen (1 atm) for 24 h, then filtered though a pad of celite and concentrated to give 4.43 g (97%) white solid: ¹H NMR (CDCl₃): δ 3.19 (s, 3H), 6.87 (d, 1H), 7.12 (t, 1H), 7.30 (t, 1H), 7.34-7.42 (m, 2H), 7.61 (t, 1H), 8.08 (d, 1H), 8.93 (d, 1H), 11.94 (s, 1H).

### Intermediate

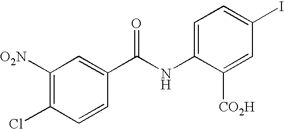

2-(4-Chloro-3-nitrobenzoylamino)-5-iodobenzoic acid. A suspension of 2-aminobenzoic acid (9.71 g, 37 mmol) and Et₃N (16 mL, 116 mmol) in CH₂Cl₂ (260 mL) was added 3-nitrobenzoyl chloride (8.54 g, 39 mmol), and the reaction mixture was stirred under argon. After 12 h the reaction mixture was concentrated and the residue was partitioned between 3% HCl and CH₂Cl₂. The aqueous phase was extracted with CH₂Cl₂, the combined organic phases were concentrated and the precipitate was recrystallized from acetonitrile to give 7.38 g (45% yield): LC/MS (an10n8): Rt 3.97 min, m/z 444.8 [M−H]⁻; ¹H NMR (DMSO-d₆): δ 7.96 (dd, J=8.9, 2.3 Hz, 1H), 8.01 (d, J=8.5 Hz, 1H), 8.16 (dd, J=8.3, 2.1 Hz, 1H), 8.26 (d, J=2.3 Hz, 1H), 8.32 (d, J=8.7 Hz, 1H), 8.53 (d, J=2.1 Hz, 1H), 12.11 (s, 1H).

### Intermediate

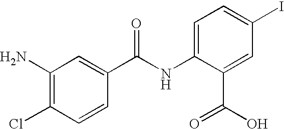

2-(3-Amino-4-chlorobenzoylamino)-5-iodobenzoic acid. A solution of 2-(chloro-3-nitrobenzoylamino)-5-iodobenzoic acid (4.0 g, 9.0 mmol) in acetic acid (20 mL) at 15° C. was added dropwise SnCl₂2H₂O (5.1 g, 23 mmol) in conc. HCl (5.76 mL), and the reaction mixture was stirred at room temperature. After 48 h the reaction mixture was neutralized with 2.5 M NaOH and extracted with EtOAc. The organic phase was washed with brine, dried (MgSO₄) and concentrated to give 3.05 g (81% yield): LC/MS (an10n8): Rt 3.64 min, m/z 414.9 [M−H]⁻; ¹H NMR (DMSO-d₆): δ 5.62 (s, 2H), 7.16 (dd, J=8.3, 1.5 Hz, 1H), 7.31 (d, J=8.3 Hz, 1H), 7.44 (d, J=1.9 Hz, 1H), 7.67 (dd, J=8.7, 2.3 Hz, 1H), 8.39 (d, J=2.3 Hz, 1H), 8.47 (dd, J=8.7, 0.9 Hz, 1H).

### Intermediate

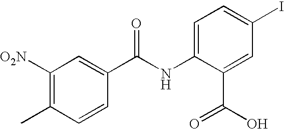

5-Iodo-2-(4-methyl-3-nitrobenzoylamino)benzoic acid. Prepared by a method analogous to the one described for B-5 to give 7.89 g (49% yield): LC/MS (an 10n8): Rt 3.65 min, m/z 424.9 [M−H]⁻.

### B1

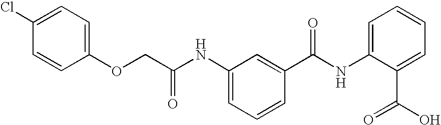

2-{3-[2-(4-Chlorophenoxy)acetylamino]benzoylamino}benzoic acid. Prepared according to GP1: purification of 47.7 mg crude pr. through silica with ([MeOH w/5% NH₄OH]:EtOAc, 1:5) as eluent gave 22 mg of the title product: LC/MS (an10n8): Rt 2.93 min, m/z 423.0 [M−H]⁻; ¹H NMR (DMSO-d₆): δ 4.68 (s, 2H), 6.91-6.97 (m, 2H), 7.02-7.08 (m, 1H), 7.18-7.25 (m, 2H), 7.28-7.40 (m, 3H), 7.51-7.59 (m, 1H), 7.64-7.71 (m, 2H), 7.86-7.92 (m, 1H), 8.04-8.09 (m, 1H), 8.33 (s, 1H), 8.72 (d, J=8.5 Hz, 1H), 10.39 (s, 1H), 12.18 (s, 1H).

### B2

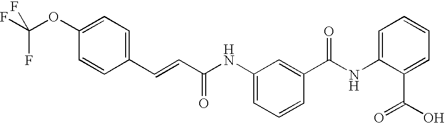

2-{3-[(E)-3-(4-Trifluoromethoxyphenyl)acryloylamino]benzoylamino}benzoic acid. Prepared according to GP1 to give 56.7 mg crude product. Recrystallization from 0.5 ml (EtOAc:[MeOH w/5% NH₄OH], 5:1) gave 32.8 mg yield (58%): LC/MS (an10n8): Rt 3.19 min, m/z 469 [M−H]⁻; ¹H NMR (DMSO-d₆): δ 6.93 (d, J=15.6 Hz, 1H), 6.99-7.05 (m, 1H), 7.32-7.37 (m, 1H), 7.44 (d, J=8.7 Hz, 2H), 7.46-7.52 (m, 1H), 7.66 (d, J=15.8 Hz, 1H), 7.73-7.76 (m, 1H), 7.81 (d, J=6.6 Hz, 2H), 8.00-8.07 (m, 2H), 8.28 (s, 1H), 8.68 (d, J=5.3 Hz, 1H), 10.60 (s, 1H), 15.27 (s, 1H).

### B3

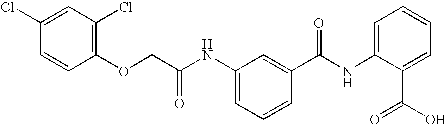

2-{3-[2-(2,4-Dichlorophenoxy)acetylamino]benzoylamino}-benzoic acid. Prepared according to GP2: LC/MS (an10p8): Rt 3.5 min, m/z 458.5 [M+1]⁺.

### B4

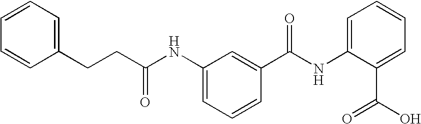

2-[3-(3-Phenylpropionylamino)benzoylamino]benzoic acid. Prepared according to GP2: LC/MS (an 10p8): Rt 2.9 min, m/z 388.6 [M+H]⁺.

### B5

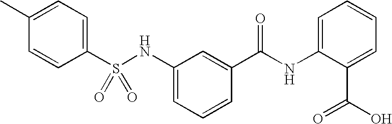

2-[3-(Toluene-4-sulfonylamino)benzoylamino]benzoic acid. Prepared according to GP1: LC/MS (an10p8): Rt 2.44 min, m/z 410.6 [M+1]⁺.

### B6

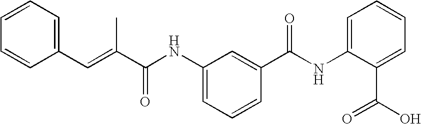

2-[3-((E)-2-Methyl-3-phenylacryloylamino)benzoylamino]benzoic acid. Prepared according to GP2: LC/MS (an 10p8): Rt 3.10 min, m/z 400.6 [M+1]⁺.

### B7

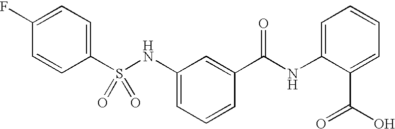

2-[3-(4-Fluoro-benzenesulfonylamino)benzoylamino]benzoic acid. Prepared according to GP1: LC/MS (an10n8): Rt 0.52 min, m/z 413.0 [M−1]⁻.

### B8

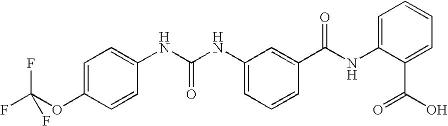

2-{3-[3-(4-Trifluoromethoxyphenyl)ureido]benzoylamino}benzoic acid. Prepared according to GP1: LC/MS (an10n8): Rt 3.98 min, m/z 458.0 [M−H]⁻.

### B9

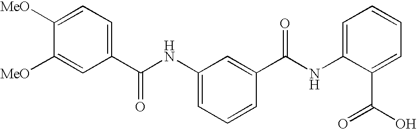

2-[3-(3,4-Dimethoxybenzoylamino)benzoylamino]benzoic acid. Prepared according to GP1: LC/MS (an 10n8): Rt 2.71 min, m/z 419.0 [M−H]⁻.

### B10

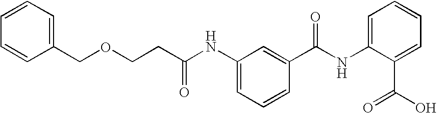

2-[3-(2-Benzyloxyacetylamino)benzoylamino]benzoic acid. Prepared according to GP1: LC/MS (an10n8): Rt 2.78 min, m/z 403.1 [M−H]⁻.

### B11

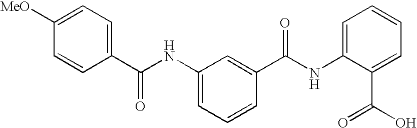

2-[3-(4-Methoxybenzoylamino)benzoylamino]benzoic acid. Prepared according to GP1: LC/MS (an 10n8): Rt 2.61 min, m/z 389.0 [M−H]⁻.

### B12

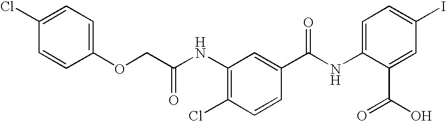

2-{4-Chloro-3-[2-(4-chloro-phenoxy)acetylamino]benzoylamino}-5-iodobenzoic acid. Prepared according to GP2: LC/MS (an 10p8): Rt 2.50 min, m/z 584.2 [M+H]⁺.

### Intermediate

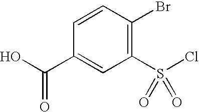

4-Bromo-3-chlorosulfonylbenzoic acid. Prepared from 4-bromobenzoic acid (20.1 g, 100 mmol) according to GP3 to give 25.8 g (86%) of the title compound.

### Intermediate

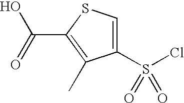

4-Chlorosulfonyl-3-methylthiophene-2-carboxylic acid. Prepared from 3-methylthiophene-2-carboxylic acid (10.0 g, 70 mmol) and chlorosulfonic acid (38 mL, 560 mmol) according to GP3 to give 13.2 g (79%) of the title compound.

### B13

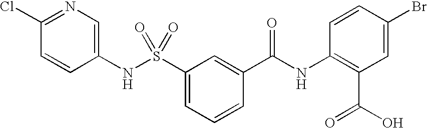

5-Bromo-2-[3-(6-chloropyridin-3-ylsulfamoyl)benzoylamino]benzoic acid. Prepared from 3-chlorosulfonylbenzoic acid, 5-amino-2-chloropyridine, and methyl 2-amino-5-bromobenzoate according to GP4, GP5, GP6 and GP7 to give 12.9 mg (overall yield: 25%) of the title compound: LC/MS (an 10p8): Rt 2.84 min, m/z 511 [M+H]⁺; ¹H NMR (DMSO): δ 7.44 (d, 1H), 7.64-7.67 (dd, 1H), 7.83 (m, 2H), 8.03 (d, 1H), 8.13-8.18 (m, 3H), 8.39 (s, 1H), 8.55 (d, 1H), 11.08 (s, 1H), 12.15 (s, 1H)

### B14

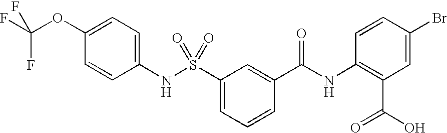

5-Bromo-2-[3-(4-trifluoromethoxyphenylsulfamoyl)benzoylamino]benzoic acid. Prepared from 3-chlorosulfonylbenzoic acid, 4-(trifluoromethoxy)aniline and methyl 2-amino-5-bromobenzoate according to GP4, GP5, GP6 and GP7: LC/MS (an10p8): Rt 3.79 min, m/z 558 [M+H]⁺.

### B15

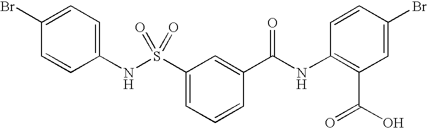

5-Bromo-2-[3-(4-bromophenylsulfamoyl)benzoylamino]benzoic acid. Prepared from 3-chlorosulfonylbenzoic acid, 4-bromoaniline and methyl 2-amino-5-bromobenzoate according to GP4, GP5, GP6 and GP7: ¹H NMR (DMSO): δ 7.08 (d, 2H), 7.44 (d, 2H), 7.84 (m, 2H), 7.98 (d, 1H), 8.15-8.19 (m, 2H), 8.39 (s, 1H), 8.59 (d, 1H), 10.64 (s, 1H), 12.18 (s, 1H).

### B16

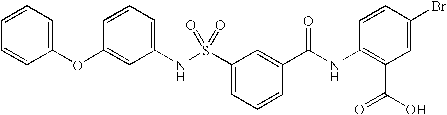

5-Bromo-2-[3-(3-phenoxyphenylsulfamoyl)benzoylamino]benzoic acid. Prepared from 3-chlorosulfonylbenzoic acid, 3-phenoxyaniline and methyl 2-amino-5-bromobenzoate according to GP4, GP5, GP6 and GP7: LC/MS (an10p8) Rt 4.35 min, m/z 566 [M+H]⁺.

### B17

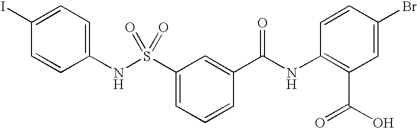

5-Bromo-2-[3-(4-iodophenylsulfamoyl)benzoylamino]benzoic acid. Prepared from 3-chlorosulfonylbenzoic acid, 4-iodoaniline and methyl 2-amino-5-bromobenzoate according to GP4, GP5, GP6 and GP7: LC/MS (an10p8) Rt 3.93 min, m/z 601 [M+H]⁺.

### B18

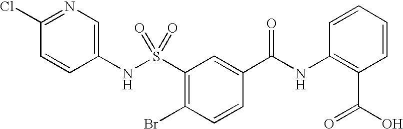

2-[4-Bromo-3-(6-chloropyridin-3-ylsulfamoyl)benzoylamino]benzoic acid. Prepared from 4-bromo-3-chlorosulfonylbenzoic acid, 5-amino-2-chloropyridine, and methyl anthranilate according to GP4, GP5, GP6, and GP7 to give 28.4 mg (yield: 13%) of the title compound: LC/MS (an10p8): Rt 1.92 min, m/z 510 [M+H]⁺; ¹H NMR (DMSO-d₆): δ 7.27 (t, 1H), 7.46 (d, 2H), 7.63 (dd, 1H), 7.70 (t, 1H), 8.09-8.11 (m, 4H), 8.20 (d, 1H), 8.64 (d, 1H), 8.68 (s, 1H), 12.39 (s, 1H).

### B19

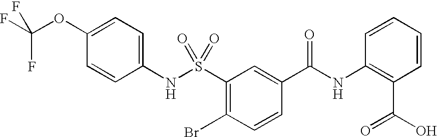

2-[4-Bromo-3-(4-trifluoromethoxyphenylsulfamoyl)benzoylamino]benzoic acid. Prepared from 4-bromo-3-chlorosulfonylbenzoic acid, 4-(trifluoromethoxy)aniline and methyl anthranilate according to GP4, GP5, GP6, and GP7: LC/MS (an10p8): Rt 4.64 min, m/z 559 [M+H]⁺.

### B20

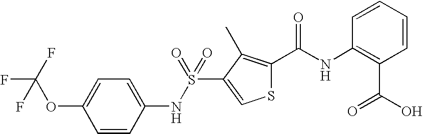

2-[3-Methyl-4-(4-trifluoromethoxyphenylsulfamoyl)thiophene-2-carbonylamino]benzoic acid. Prepared from 4-chlorosulfonyl-3-methylthiophene-2-carboxylic acid, 4-trifluoromethoxyphenylamine and methyl anthranilate according to GP4, GP5, GP6, and GP7 to give 42.2 mg (yield: 20%) of the title compound: LC/MS (an10n8) Rt 4.45 min, m/z 499 [M−H]⁻; ¹H NMR (DMSO): δ 2.67 (s, 3H), 7.26 (m, 6H), 7.65 (t, 1H), 8.03 (d, 1H), 8.52 (s, 2H), 10.82 (s, 1H), 11.92 (s, 1H).

### B21

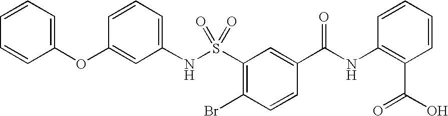

2-[4-Bromo-3-(3-phenoxyphenylsulfamoyl)benzoylamino]benzoic acid. Prepared from 4-bromo-3-chlorosulfonylbenzoic acid, 3-phenoxyaniline and methyl anthranilate according to GP4, GP5, GP6, and GP7: LC/MS (an10n8) Rt 4.51 min, m/z 565 [M−H]⁻.

### B22

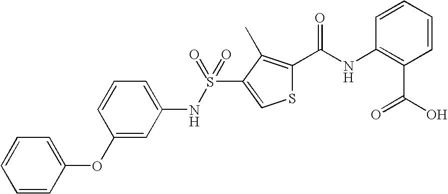

2-[3-Methyl-4-(3-phenoxyphenylsulfamoyl)thiophene-2-carbonylamino]-benzoic acid. Prepared from 4-chlorosulfonyl-3-methylthiophene-2-carboxylic acid, 3-phenoxyaniline and methyl anthranilate according to GP4, GP5, GP6, and GP7: LC/MS (an 10n8) Rt 4.25 min, m/z 507 [M−H]⁻.

### B23

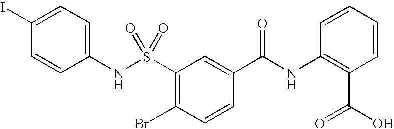

2-[4-Bromo-3-(4-iodophenylsulfamoyl)benzoylamino]benzoic acid. Prepared from 4-bromo-3-chlorosulfonylbenzoic acid, 4-iodoaniline and methyl anthranilate according to GP4, GP5, GP6, and GP7: LC/MS (an10n8) Rt 4.33 min, m/z 601 [M−H]⁻

### B24

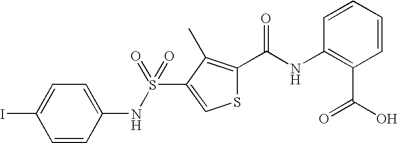

2-[4-(4-Iodophenylsulfamoyl)-3-methylthiophene-2-carbonylamino]benzoic acid. Prepared from 4-chlorosulfonyl-3-methylthiophene-2-carboxylic acid, 4-iodoaniline and methyl anthranilate according to GP4, GP5, GP6, and GP7: LC/MS (an 10n8) Rt 4.04 min, m/z 541 [M−H]⁻.

### B25

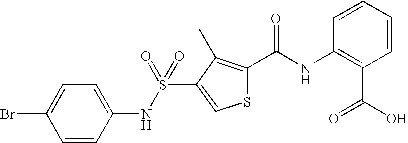

2-[4-(4-Bromophenylsulfamoyl)-3-methylthiophene-2-carbonylamino]-benzoic acid. Prepared from 4-chlorosulfonyl-3-methylthiophene-2-carboxylic acid, 4-bromoaniline and methyl anthranilate according to GP4, GP5, GP6, and GP7: LC/MS (an10p8): Rt 3.86 min, m/z 497 [M+H]⁺.

### B26

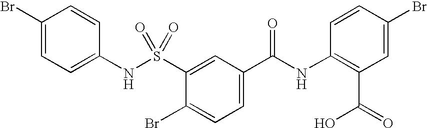

5-Bromo-2-[4-bromo-3-(4-bromophenylsulfamoyl)benzoylamino]benzoic acid. Prepared from 4-bromo-3-chlorosulfonylbenzoic acid, 4-bromoaniline and methyl 2-amino-5-bromobenzoate according to GP4, GP5, GP6, and GP7: LC/MS (an10p8): Rt 3.84 min, m/z 634 [M+H]⁺

### B27

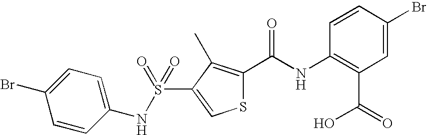

5-Bromo-2-[4-(4-bromophenylsulfamoyl)-3-methylthiophene-2-carbonylamino]benzoic acid. Prepared from 4-chlorosulfonyl-3-methylthiophene-2-carboxylic acid, 4-bromoaniline and methyl 2-amino-5-bromobenzoate according to GP4, GP5, GP6, and GP7: LC/MS (an10p8): Rt 3.67 min, m/z 575 [M+H]⁺

### B28

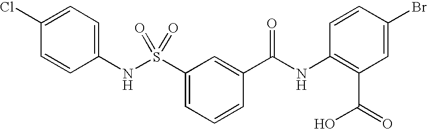

5-Bromo-2-[3-(4-chlorophenylsulfamoyl)benzoylamino]benzoic acid. Prepared from 3-chlorosulfonylbenzoic acid, 4-chloroaniline and methyl 2-amino-5-bromobenzoate according to GP4, GP5, GP6, and GP7: LC/MS (an 10n8) Rt 2.90 min, m/z 509 [M−H]⁻.

### B29

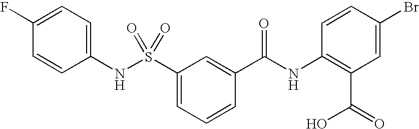

5-Bromo-2-[3-(4-fluorophenylsulfamoyl)benzoylamino]benzoic acid. Prepared from 3-chlorosulfonylbenzoic acid, 4-fluoroaniline and methyl 2-amino-5-bromobenzoate according to GP4, GP5, GP6, and GP7: LC/MS (an10n8): Rt 2.62 min, m/z 493 [M−H]⁻.

### B30

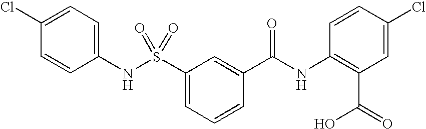

5-Chloro-2-[3-(4-chlorophenylsulfamoyl)benzoylamino]benzoic acid. Prepared from 3-chlorosulfonylbenzoic acid, 4-chloroaniline and methyl 2-amino-5-chlorobenzoate according to GP4, GP5, GP6, and GP7: LC/MS (an10n8) Rt 2.93 min, m/z 463 [M−H]⁻.

### B31

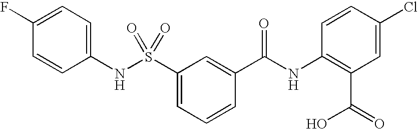

5-Chloro-2-[3-(4-fluorophenylsulfamoyl)benzoylamino]benzoic acid. Prepared from 3-chlorosulfonylbenzoic acid, 4-fluoroaniline and methyl 2-amino-5-chlorobenzoate according to GP4, GP5, GP6, and GP7: LC/MS (an10n8) Rt 2.59 min, m/z 447 [M−H]⁻.

### Biological Assays

### Materials and Methods

Generation/origin of the cDNA Constructs. The coding sequence of the human CRTH2 receptor (genbank accession no NM_{—}004778) was amplified by PCR from a human hippocampus cDNA library and inserted into the pcDNA3.1(+) expression vector (invitrogen) via 5′ HindIII and 3′ EcoRI. The sequence identity of the construct was verified by restriction endonuclease digests and sequencing in both directions on an ABI Prism (Applied Biosystems, Foster City, Calif.).

Sequence ID CRTH2 (protein sequence):
MSANATLKPLCPILEQMSRLQSHSNTSIRYIDHAAVLLHGLASLLGLVEN
GVILFVVGCRMRQTVVTTWVLHLALSDLLASASLPFFTYFLAVGHSWELG
TTFCKLHSSIFFLNMFASGFLLSAISLDRCLQVVRPVWAQNHRTVAAAHK
VCLVLWALAVLNTVPYFVFRDTISRLDGRIMCYYNVLLLNPGPDRDATCN
SRQAALAVSKFLLAFLVPLAIIASSHAAVSLRLQHRGRRRPGRFVRLVAA
VVAAFALCWGPYHVFSLLEARAHANPGLRPLVWRGLPFVTSLAFFNSVAN
PVLYVLTCPDMLRKLRRSLRTVLESVLVDDSELGGAGSSRRRRTSSTARS
ASPLALCSRPEEPRGPARLLGWLLGSCAASPQTGPLNPALSSTSS
Sequence ID CRTH2 (nucleotide sequence):
atgtcggc caacgccaca ctgaagccac tctgccccat
cctggagcag atgagccgtc tccagagcca cagcaacacc
agcatccgct acatcgacca cgcggccgtg ctgctgcacg
ggctggcctc gctgctgggc ctggtggaga atggagtcat
cctcttcgtg gtgggctgcc gcatgcgcca gaccgtggtc
accacctggg tgctgcacct ggcgctgtcc gacctgttgg
cctctgcttc cctgcccttc ttcacctact tcttggccgt
gggccactcg tgggagctgg gcaccacctt ctgcaaactg
cactcctcca tcttctttct caacatgttc gccagcggct
tcctgctcag cgccatcagc ctggaccgct gcctgcaggt
ggtgcggccg gtgtgggcgc agaaccaccg caccgtggcc
gcggcgcaca aagtctgcct ggtgctttgg gcactagcgg
tgctcaacac ggtgccctat ttcgtgttcc gggacaccat
ctcgcggctg gacgggcgca ttatgtgcta ctacaatgtg
ctgctcctga acccggggcc tgaccgcgat gccacgtgca
actcgcgcca ggcggccctg gccgtcagca agttcctgct
ggccttcctg gtgccgctgg cgatcatcgc ctcgagccac
gcggccgtga gcctgcggtt gcagcaccgc ggccgccggc
ggccaggccg cttcgtgcgc ctggtggcag ccgtcgtggc
cgccttcgcg ctctgctggg ggccctacca cgtgttcagc
ctgctggagg cgcgggcgca cgcaaacccg gggctgcggc
cgctcgtgtg gcgcgggctg cccttcgtca ccagcctggc
cttcttcaac agcgtggcca acccggtgct ctacgtgctc
acctgccccg acatgctgcg caagctgcgg cgctcgctgc
gcacggtgct ggagagcgtg ctggtggacg acagcgagct
gggtggcgcg ggaagcagcc gccgccgccg cacctcctcc
accgcccgct cggcctcccc tttagctctc tgcagccgcc
cggaggaacc gcggggcccc gcgcgtctcc tcggctggct
gctgggcagc tgcgcagcgt ccccgcagac gggccccctg
aaccgggcgc tgagcagcac ctcgagttag

Cell Culture and Transfection. COS-7 cells were grown in Dulbecco's modified Eagle's medium (DMEM) 1885 supplemented with 10% fetal bovine serum, 100 units/ml penicillin, 1000 pg/ml streptomycin, and kept at 37° C. in a 10% CO₂ atmosphere. HEK293 cells were maintained in Minimum Essential medium (MEM) supplemented with 10% (v/v) heat inactivated fetal calf serum (HIFCS), 2 mM Glutamax™-I, 1% non essential amino acids (NEAA), 1% sodium pyruvate and 10 μg/ml gentamicin. For binding experiments, COS7 cells were transiently transfected with the CRTH2 receptor using a calcium phosphate-DNA coprecipitation method with the addition of chloroquine (as described by Holst B, Hastrup H, Raffetseder U, Martini L, Schwartz T W. J Biol. Chem. 2001 Jun. 8; 276(23):19793-9.)

Binding assay. 24 h after transfection COS-7 cells were seeded into 96 well plates at a density of 30.000 cells/well. Competition binding experiments on whole cells were then performed about 18-24 h later using 0.1 nM [³H]PGD2 (NEN, 172 Ci/mmol) in a binding buffer consisting of HBSS (GIBCO) and 10 mM HEPES. Competing ligands were diluted in DMSO which was kept constant at 1% (v/v) of the final incubation volume. Total and nonspecific binding were determined in the absence and presence of 10 μM PGD2. Binding reactions were routinely conducted for 3 h at 4° C. and terminated by 2 washes (100 μl each) with ice cold binding buffer. Radioactivity was determined by liquid scintillation counting in a TOPCOUNTER (Packard) following over night incubation in Microscint 20. Stable HEK293 cells were seeded at a density of 30.000 cells/well 18-24 h prior to the binding assay which was performed essentially as described for COS7 cells above. Determinations were made in duplicates.

### Materials

Tissue culture media and reagents were purchased from the Gibco invitrogen corporation (Breda, Netherlands). PGD2 was obtained from Cayman and [3H]PGD2 from NEN.

### Data Analysis

Curve analysis was performed with the GraphPadPrism software 3.0 (Graphpad Prism Inc., San Diego, USA) and IC₅₀ values were calculated as a measure of the antagonistic potencies.

### Biological Data:

Compounds of Series B were tested in the receptor binding assay described below, and their IC50s were assessed. The compounds are grouped in three classes:

A: IC₅₀ value lower than 0.5 μM
B: IC₅₀ value between 0.5 μM and 5 μM
C: IC₅₀ value higher than 5 μM

Tables 1 and 2 give the results for compounds synthesised as described above, and Tables 3, and 4 give the results for compounds acquired commercially.

TABLE 1

| Cmp | Structure | IC₅₀ |
|---|---|---|
| B1 | 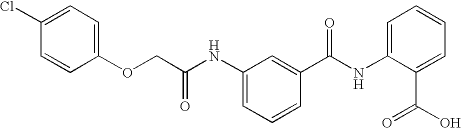 | A |
| B2 | 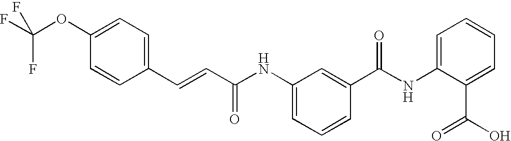 | A |
| B3 | 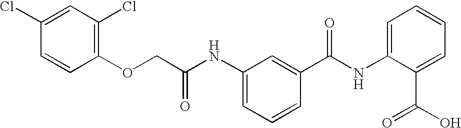 | A |
| B4 | 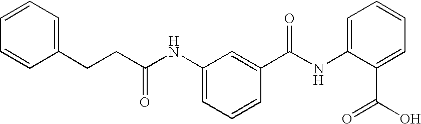 | B |
| B5 | 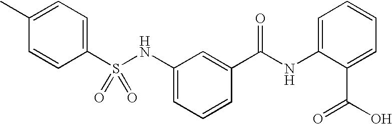 | B |
| B6 | 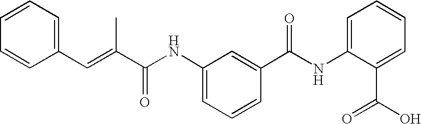 | B |
| B7 | 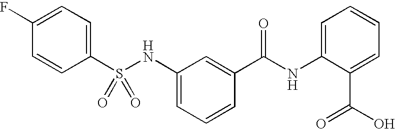 | B |
| B8 | 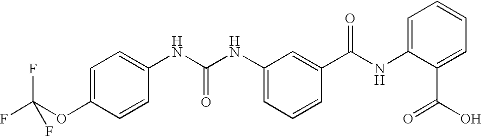 | B |
| B9 | 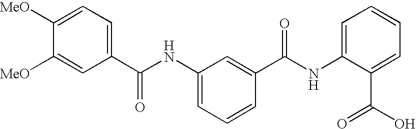 | B |
| B10 | 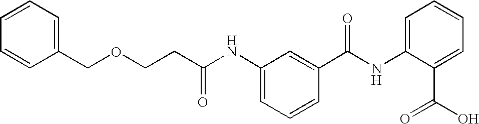 | B |
| B11 | 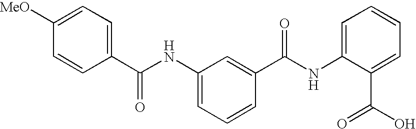 | B |
| B12 | 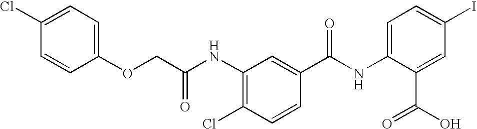 | B |

TABLE 2

| Cmp | Structure | IC₅₀ |
|---|---|---|
| B13 | 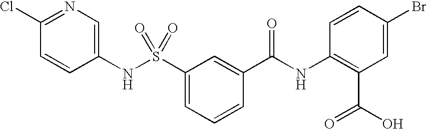 | B |
| B14 | 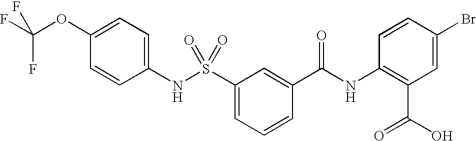 | B |
| B15 | 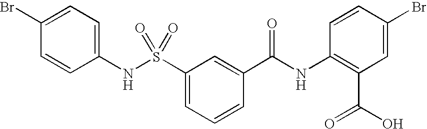 | A |
| B16 | 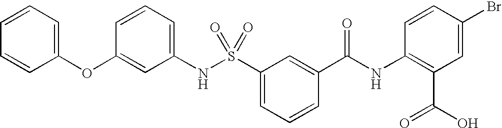 | A |
| B17 | 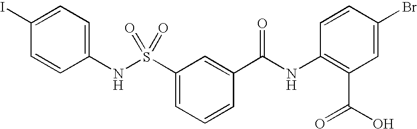 | A |
| B18 | 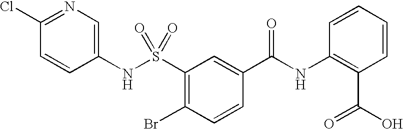 | B |
| B19 | 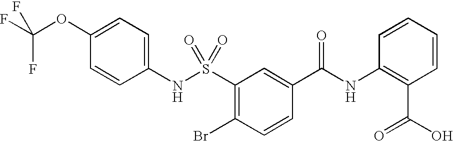 | A |
| B20 | 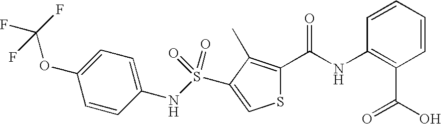 | A |
| B21 | 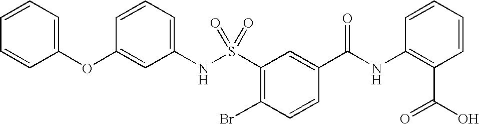 | A |
| B22 | 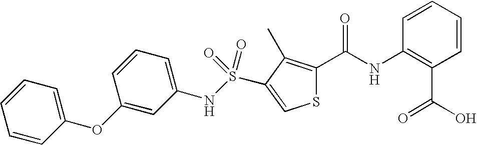 | B |
| B23 | 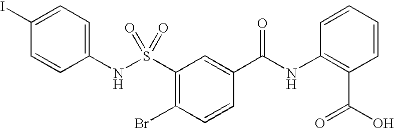 | A |
| B24 | 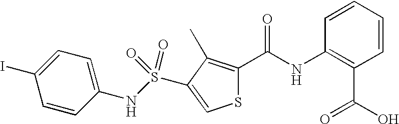 | A |
| B25 | 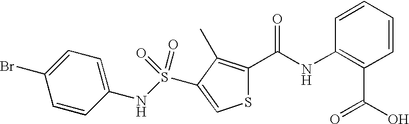 | A |
| B26 | 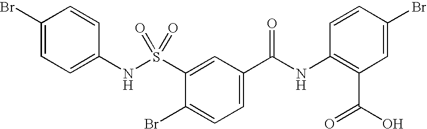 | A |
| B27 | 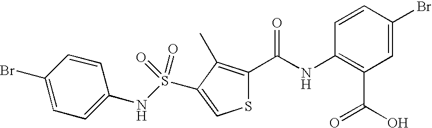 | A |
| B28 | 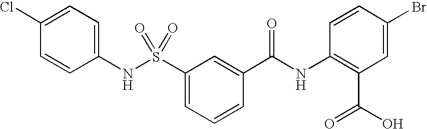 | A |
| B29 | 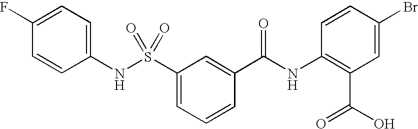 | A |
| B30 | 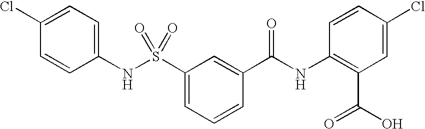 | A |
| B31 | 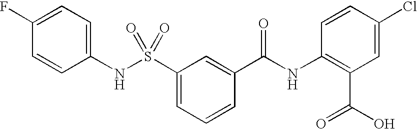 | A |

TABLE 3

| Cmp | Structure | IC₅₀ |
|---|---|---|
| B32 | 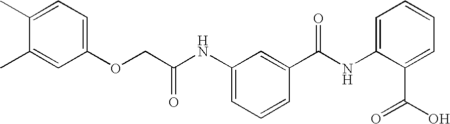 | A |
| B33 | 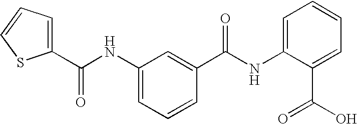 | B |
| B34 | 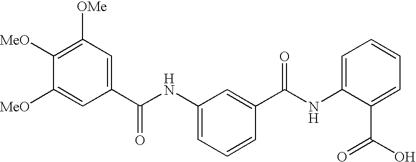 | B |
| B35 | 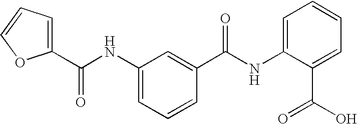 | C |
| B36 | 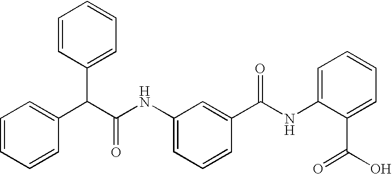 | A |
| B37 | 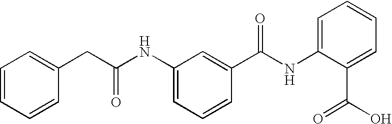 | B |
| B38 | 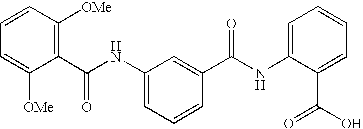 | B |
| B39 | 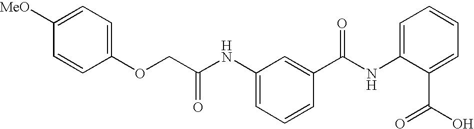 | B |
| B40 | 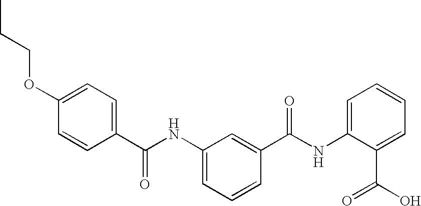 | A |
| B41 | 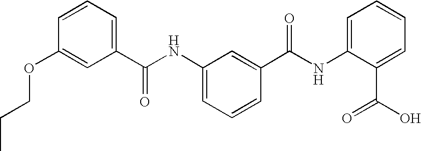 | A |
| B42 | 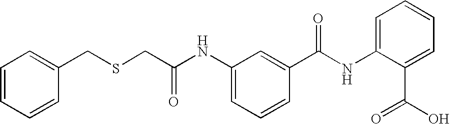 | A |
| B43 | 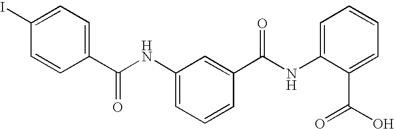 | A |
| B44 | 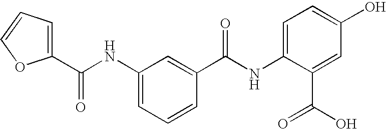 | B |
| B45 | 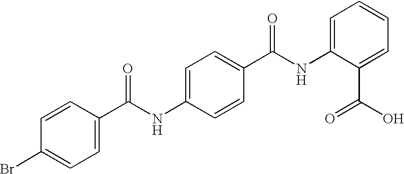 | B |

TABLE 4

| Cmp | Structure | IC₅₀ |
|---|---|---|
| B46 | 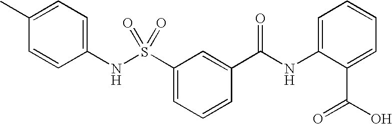 | B |
| B47 | 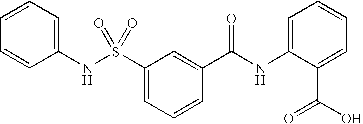 | B |
| B48 | 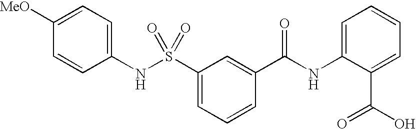 | B |
| B49 | No compound B49 | |
| B50 | 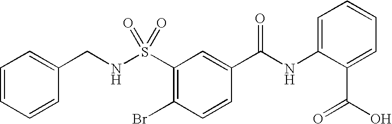 | B |
| B51 | 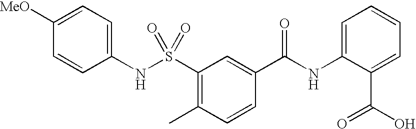 | B |
| B52 | 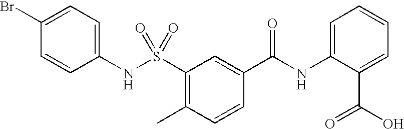 | A |
| B53 | 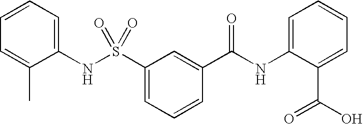 | B |
| B54 | 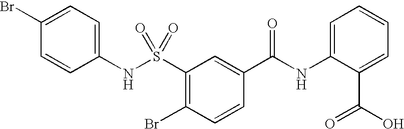 | A |
| B55 | 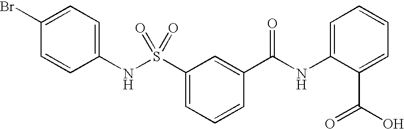 | A |
| B56 | 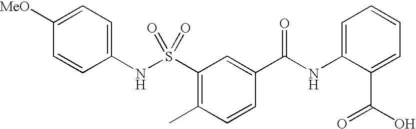 | B |
| B57 | 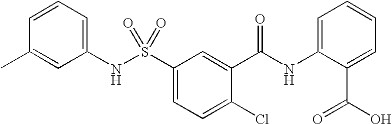 | B |
| B58 | 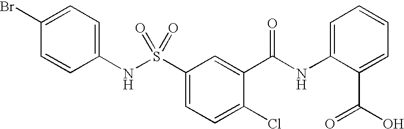 | B |
| B59 | 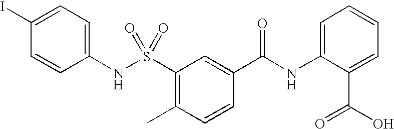 | A |
| B60 | 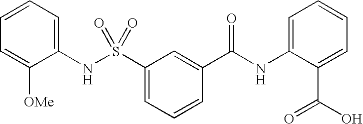 | B |
| B61 | 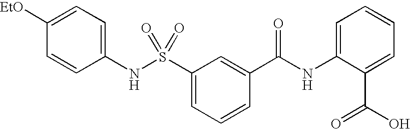 | B |
| B62 | 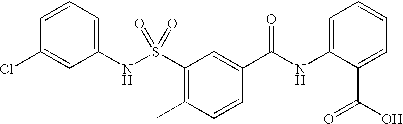 | A |
| B63 | 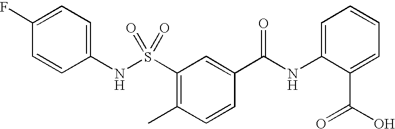 | B |
| B64 | 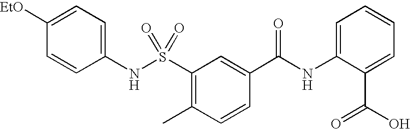 | B |
| B65 | 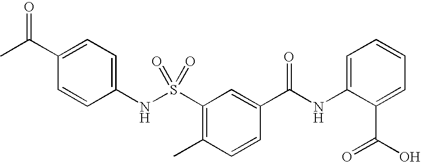 | C |
| B66 | 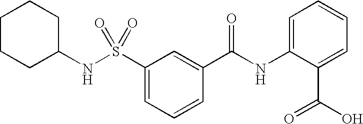 | B |
| B67 | 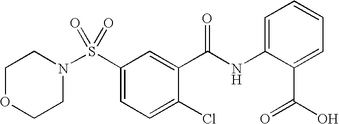 | C |
| B68 | 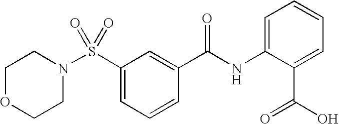 | C |
| B69 | 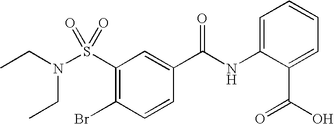 | B |
| B70 | 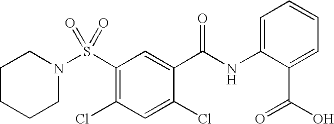 | A |
| B71 | 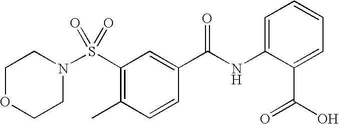 | B |
| B72 | 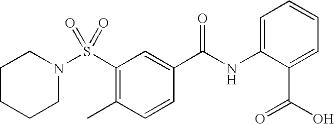 | A |
| B73 | 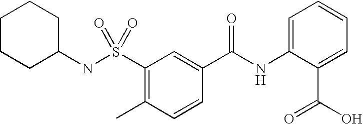 | B |
| B74 | 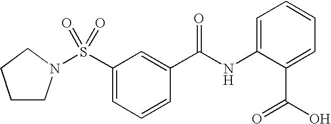 | C |
| B75 | 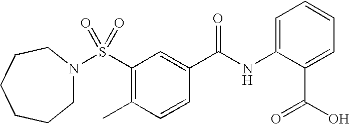 | B |
| B76 | 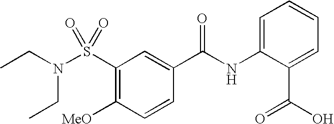 | B |
| B77 | 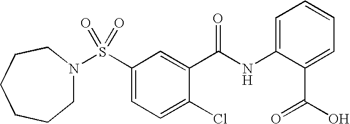 | B |
| B78 | 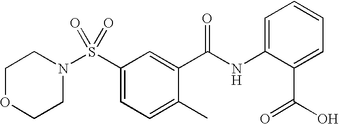 | C |
| B79 | 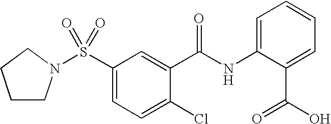 | B |
| B80 | 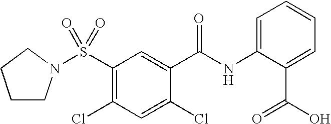 | B |
| B81 | 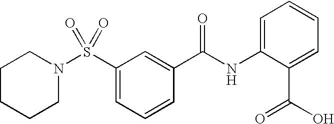 | B |

1. A method of treatment of disease responsive to modulation of CRTH2 receptor activity comprising administering to a subject suffering such disease and effective amount of a compound of formula (D) or a salt, hydrate or solvate thereof in the manufacture of a composition for the treatment of disease responsive to modulation of CRTH2 receptor activity: 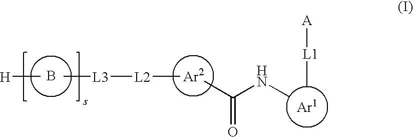
wherein:
A represents a carboxyl group —COOH, or a carboxyl bioisostere,
L1 is a bond, —CH₂—, —OCH₂—, —CH₂CH₂— or —CH═CH—;
L2 is CONH—, —NHCO—, SO₂NR¹—, —NR¹SO₂ wherein R¹ is hydrogen or C₁-C₃ alkyl, or a divalent radical of formula (X) or (Y), 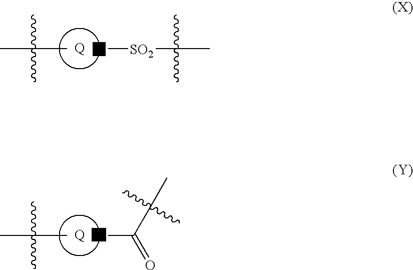
wherein ring Q is a non aromatic heterocyclic ring containing 5 to 7 ring atoms, including the nitrogen shown;
L3 is a divalent radical of formula -(Alk¹)ₘ-(Z)ₙ-(Alk²)ₚ wherein
m, n and p are independently 0 or 1,
Alk¹ and Alk² are independently optionally substituted straight or branched chain C₁-C₃ alkylene or C₂-C₃ alkenylene radicals which may contain a compatible —O—, —S— or —NR— link wherein R is hydrogen or C₁-C₃ alkyl, and
Z is —O—; —S—; —C(═O)—; —SO₂—; —SO—; or —NR—, wherein R is hydrogen or C₁-C₃ alkyl; or a divalent 5- or 6-membered monocyclic carbocyclic or heterocyclic radical,
ring Ar¹ is an optionally substituted divalent phenyl radical or divalent 5- or 6-membered monocyclic heteroaryl radical, in which L1 and the H[B]ₛL3L2Ar²CONH-radical are linked to adjacent ring carbon atoms;
ring Ar² is an optionally substituted 1,3-phenylene radical, or an optionally substituted divalent 5- or 6-membered monocyclic heteroaryl radical, in which AL1Ar¹NHCO-radical and the H[B]ₛL3L2-radical are linked to ring carbon atoms which are not in ortho relationship;
ring B is as defined for Ar², or an optionally substituted cycloalkyl, N-pyrrolidinyl, N-piperidinyl or N-azepinyl ring; and
s is 0 or 1.

2. (canceled)

3. A method as claimed in claim 1 wherein, in the compound (I), Ar¹ is an optionally substituted phenyl ring,

4. A method as claimed in claim 1 wherein, in the compound (I), ring Ar¹ is selected from the group consisting of: 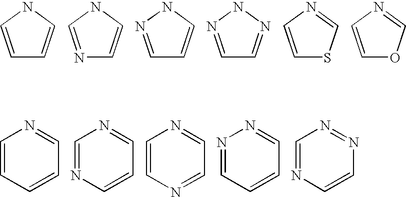
any of which being optionally substituted.

5. A method as claimed in claim 1 wherein, in the compound (I), any optional substituents in ring Ar¹ are selected from fluoro, chloro, bromo, iodo, cyano, nitro, trifluoromethyl, trifluoromethoxy, trifluoromethylthio, (C₁-C₃alkyl)SO₂—, NH₂SO₂—, (C₁-C₃alkyl)NHSO₂—, (C₁-C₃alkyl)₂NSO₂—, C₁-C₆ alkyl, C₁-C₆ alkoxy, cycloalkyl, aryl, aryloxy, aryl(C₁-C₆) and aryl(C₁-C₆ alkoxy)-.

6. A method as claimed in claim 1 wherein, in the compound (I), A is —COOH.

7. A method as claimed in any of claim 1 wherein, in the compound (I), A is a carboxyl bioisostere selected from —SO₂NHR and —P(═O)(OH)(OR) wherein R is hydrogen methyl or ethyl, —SO₂OH, —P(═O)(OH)(NH₂), —C(═O)NHCN and groups of formulae:

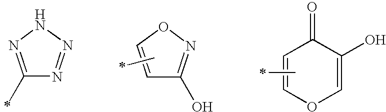

8. A method as claimed in claim 1 wherein, in the compound (I), L1 represents a bond.

9. A method as claimed in claim 1 wherein, in the compound (I), Ar² is an optionally substituted 1,3-phenylene radical.

10. A method as claimed in claim 1 wherein, in the compound (I), Ar² is selected from the following radicals, in either orientation 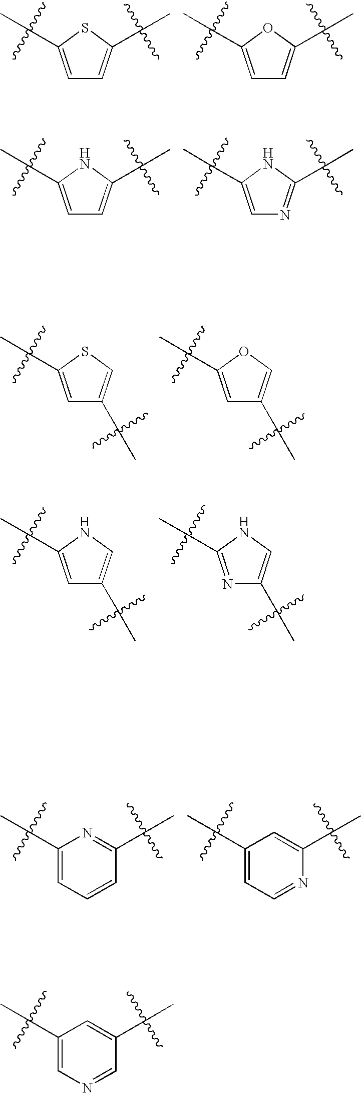
in the case of non-symmetrical radicals:

11. A method as claimed in claim 1 wherein, in the compound (I), any optional substituents in ring Ar² are selected from fluoro, chloro, bromo, iodo, cyano, nitro, trifluoromethyl, trifluoromethoxy, trifluoromethylthio, (C₁-C₃alkyl)SO₂—, NH₂SO₂—, (C₁-C₃alkyl)NHSO₂—, (C₁-C₃alkyl)₂NSO₂—, C₁-C₆ alkyl, C₁-C₆ alkoxy, cycloalkyl, aryl, aryloxy, aryl(C₁-C₆) and aryl(C₁-C₆ alkoxy)-.

12. A method as claimed in claim 1 wherein, in the compound (I), s is 1 and ring B is an optionally substituted thienyl, furanyl, pyridyl, or N-pyrrolidinyl, N-piperidinyl, N-piperazinyl, N-morpholinyl or N-azepinyl ring.

13. A method as claimed in wherein, in the compound (I), s is 1 and ring B is optionally substituted phenyl

14. A method as claimed in claim 1 wherein, in the compound (I), any optional substituents in ring B are selected from fluoro, chloro, bromo, iodo, cyano, nitro, trifluoromethyl, trifluoromethoxy, trifluoromethylthio, (C₁-C₃alkyl)SO₂—, NH₂SO₂—, (C₁-C₃alkyl)NHSO₂—, (C₁-C₃alkyl)₂NSO₂—, C₁-C₆ alkyl, C₁-C₆ alkoxy, cycloalkyl, aryl, aryloxy, aryl(C₁-C₆) or aryl(C₁-C₆ alkoxy)-.

15. A method as claimed in claim 1 wherein, in the compound (I), s is 1 and ring B is phenyl, optionally substituted by hydroxyl, mercapto, fluoro, chloro, bromo, iodo, methyl, ethyl, trifluoromethyl, methoxy, ethoxy, trifluoromethoxy, methylthio, trifluoromethylthio, dimethylamino, nitro, acetyl, or phenyl.

16. A method as claimed in claim 1 wherein, in the compound (I), L2 is a divalent radical of formula (X) or (Y) wherein the divalent radical -Q- is selected from the following

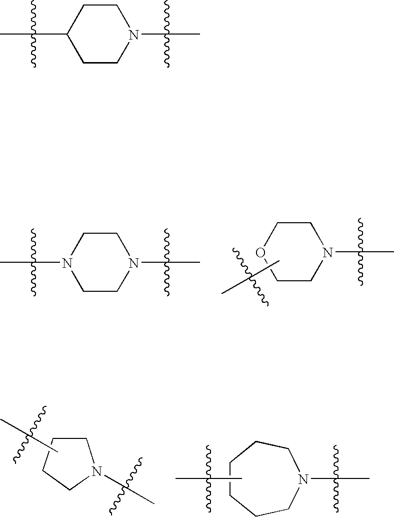

17. A method as claimed in claim 1 wherein, in the compound (I), L3 is a bond or a linker radical selected from —CH₂—, —CH(Ph)- wherein Ph is phenyl, —NR—, —CH₂CH₂—, —CH₂CH₂CH₂—, —CH₂Z-, -ZCH₂—, —CH₂CH₂Z-, —CH₂ZCH₂—
-ZCH₂CH₂—, —CH═CH—, —CH═C(CH₃)—, —CH═N—, —N═CH—, —CH═CHCH₂—, —N═CHCH₂—, —CH═NCH₂—, —CH₂CH═CH—, —CH₂Z-, -ZCH₂—, —CH₂CH₂Z-, —CH₂ZCH₂—, -ZCH₂CH₂—, —CH═CHZ-, and -ZCH═CH— wherein Z is —O—, —S— or —NR— wherein R is hydrogen or C₁-C₃ alkyl, any of which radicals being optionally substituted on one of the carbon atoms shown.

18. A method as claimed in claim 1 wherein, in the compound (I), L3 is a bond or a linker radical selected from OCH₂—, —CH₂CH₂—, —CH═CH—, —CH═C(CH₃)—, —NH—, —CH₂OCH₂CH₂—, —CH(Ph) wherein Ph is phenyl, or —CH₂SCH₂—.

19. A method as claimed in claim 1 wherein, the compound (I) has formula (II): 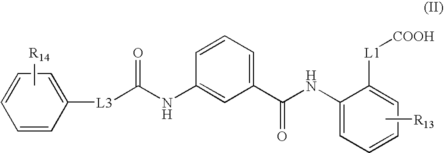
wherein L1 and L3 are as defined in claim 1, and R₁₃ and R₁₄ represent one or more optional substituents in their respective phenyl rings.

20. A method as claimed in claim 19 wherein, in the compound (II), L3 is a bond or a linker radical selected from —CH₂—, —CH(Ph)- wherein Ph is phenyl, —NR—,
—CH₂CH₂—, —CH₂CH₂CH₂—, —CH₂Z-, -ZCH₂—, —CH₂CH₂Z-, —CH₂ZCH₂—, -ZCH₂CH₂—, —CH═CH—, —CH═C(CH₃)—, —CH═N—, —N═CH—, —CH═CHCH₂—, —N═CHCH₂—, —CH═NCH₂—, —CH₂CH═CH—, —CH₂Z-, -ZCH₂—, —CH₂CH₂Z-, —CH₂ZCH₂—, -ZCH₂CH₂—, —CH═CHZ-, and -ZCH═CH— wherein Z is —O—, —S— or —NR— wherein R is hydrogen or C₁-C₃ alkyl, any of which radicals being optionally substituted on one of the carbon atoms shown.

21. A method as claimed in claim 19 wherein, in the compound (II), L3 is a bond or a linker radical selected from —OCH₂—, —CH₂CH₂—, —CH═CH—, —CH═C(CH₃)—,
—NH—, —CH₂OCH₂CH₂—, —CH(Ph) wherein Ph is phenyl, or —CH₂SCH₂—.

22. A method as claimed in claim 19 wherein, in the compound (II), optional substituents R₁₃ and R₁₄ are selected from fluoro, chloro, bromo, iodo, cyano, nitro, trifluoromethyl, trifluoromethoxy, trifluoromethylthio, (C₁-C₃alkyl)SO₂—, NH₂SO₂—, (C₁-C₃alkyl)NHSO₂—, (C₁-C₃alkyl)₂NSO₂—, C₁-C₆ alkyl, C₁-C₆ alkoxy, cycloalkyl, aryl, aryloxy, aryl(C₁-C₆) and aryl(C₁-C₆ alkoxy)-.

23. A method as claimed in claim 1 wherein the disease is one associated with elevated levels of prostaglandin D2 (PGD2) or one or more active metabolites thereof.

24. A method as claimed in claim 23 wherein the disease is an inflammatory, autoimmune, respiratory or allergy disease.

25. A method as claimed in claim 23 wherein the disease is selected from asthma, rhinitis, allergic airway syndrome, allergic rhinobronchitis, bronchitis, chronic obstructive pulmonary disease (COPD), nasal polyposis, sarcoidosis, farmer's lung, fibroid lung, cystic fibrosis, chronic cough, conjunctivitis, atopic dermatitis, Alzheimer's disease, amyotrophic lateral sclerosis, AIDS dementia complex, Huntington's disease, frontotemporal dementia, Lewy body dementia, vascular dementia, Guillain-Barre syndrome, chronic demyelinating polyradiculoneurophathy, multifocal motor neuropathy, plexopathy, multiple sclerosis, encephalomyelitis, panencephalitis, cerebellar degeneration and encephalomyelitis, CNS trauma, migraine, stroke, rheumatoid arthritis, ankylosing spondylitis, Behçet's Disease, bursitis, carpal tunnel syndrome, inflammatory bowel disease, Crohn's disease, ulcerative colitis, dermatomyositis, Ehlers-Danlos Syndrome (EDS), fibromyalgia, myofascial pain, osteoarthritis (OA), osteonecrosis, psoriatic arthritis, Reiter's syndrome (reactive arthritis), sarcoidosis, scleroderma, Sjogren's Syndrome, soft tissue disease, Still's Disease, tendinitis, polyarteritis Nodossa, Wegener's Granulomatosis, myositis (polymyositis dermatomyositis), gout, atherosclerosis, lupus erythematosus, systemic lupus erythematosus (SLE), type I diabetes, nephritic syndrome, glomerulonephritis, acute and chronic renal failure, eosinophilia fascitis, hyper IgE syndrome, sepsis, septic shock, ischemic reperfusion injury in the heart, allograft rejection after transplantations, and graft versus host disease.

26. A method as claimed in claim 23 wherein the disease selected from asthma, rhinitis, allergic airway syndrome, and allergic rhinobronchitis.

27. A compound of formula (III), or a salt, hydrate or solvate thereof: 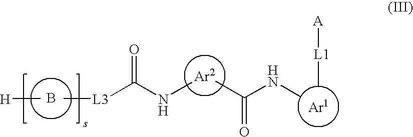
wherein: A, L1 Ar1, Ar², s and B independently are as defined in claim 1, and L3 is a linker radical selected from —CH₂CH₂—, —CH₂CH₂CH₂—, —CH₂Z-, -ZCH₂—, —CH₂CH₂Z-, —CH₂ZCH₂—, -ZCH₂CH₂—, —CH═CH—, —CH═C(CH₃)—, —CH═N—N═CH—, —CH═CHCH₂—, —N═CHCH₂—, —CH═NCH₂—, —CH₂CH═CH—, —CH₂Z-, -ZCH₂—, —CH₂CH₂Z-, —CH₂ZCH₂—, -ZCH₂CH₂—, —CH═CHZ-, and -ZCH═CH— wherein Z is —O—, —S— or —NR— wherein R is hydrogen or C₁-C₃ alkyl, any of which radicals being optionally substituted on one of the carbon atoms shown.
PROVIDED THAT
when A is —COOH, L1 is a bond, and s is 1 then (a) when Ar² is a 1,3-phenylene radical H—B-L3- is not a radical of formula (C), (D), (E) or (F): 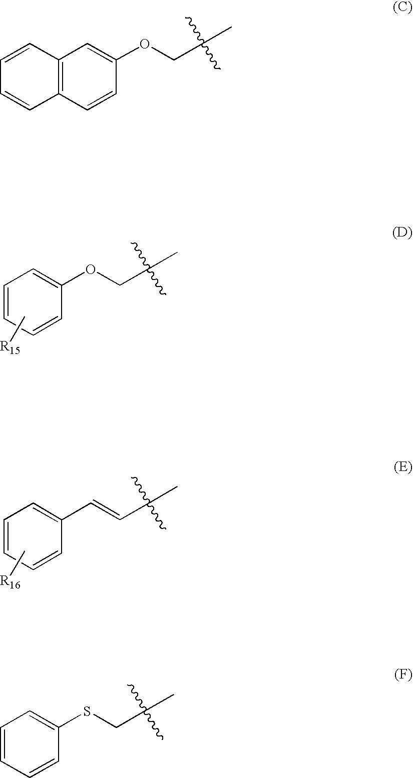
wherein R₁₅ represents hydrogen or 2- or 4-nitro, 2-, 3- or 4-methyl, 2,3-, 2,6-, or 3,4-dimethyl, 2- or 3-methoxy, 2-chloro, 4-bromo, 4-isopropyl, or 4-(1-methylpropyl), R₁₆ represents 4-nitro or 2-methoxy-5-bromo; and (b) when Ar² is a 4-methyl-1,3-phenylene radical H—B-Alk¹- is not a radical of formula (J) or (K) 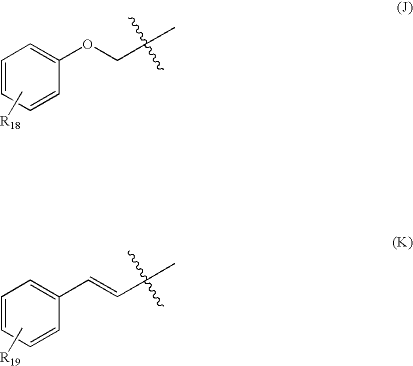
wherein R₁₈ is 2-methoxy and R₁₉ is 2-methoxy-5-bromo; and (c) when Ar² is a 4-methyl-1,3-phenylene radical H—B-Alk¹- is not a radical of formula (L)

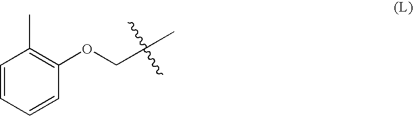

28. A compound as claimed in claim 27, and subject to the Provisos therein, having formula (II), or a salt, hydrate or solvate thereof: 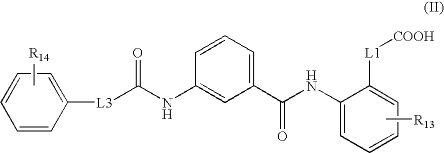
wherein L1 and L3 are as defined in claim 26, and R₁₃ and R₁₄ represent one or more optional substituents in their respective phenyl rings.

29. A compound as claimed claim 27 wherein optional substituents R₁₃ and R₁₄ are selected from fluoro, chloro, bromo, iodo, cyano, nitro, trifluoromethyl, trifluoromethoxy, trifluoromethylthio, (C₁-C₃alkyl)SO₂—, NH₂SO₂—, (C₁-C₃alkyl)NHSO₂—, (C₁-C₃alkyl)₂NSO₂—, C₁-C₆ alkyl, C₁-C₆ alkoxy, cycloalkyl, aryl, aryloxy, aryl(C₁-C₆)— and aryl(C₁-C₆ alkoxy)-.

30. A compound as claimed in claim 27 wherein L1 is a bond.

31. A pharmaceutical composition comprising a compound as claimed in any of claim 27, together with a pharmaceutically acceptable carrier.
